(19) United States
(12) Patent Application Publication
(10) Pub. No.: US 2008/0318244 A1
(43) Pub. Date: Dec. 25, 2008

(54) Large-scale parallel nucleic acid analysis method

(75) Inventors: Hiroko Matsunaga, Koganei (JP); Hideki Kambara, Hachioji (JP); Tomoharu Kajiyama, Higashiyamato (JP)

Correspondence Address: REED SMITH LLP, Suite 1400, 3110 Fairview Park Drive, Falls Church, VA, 22042, US

(73) Assignee: Hitachi, Ltd.

(21) Appl. No.: 12/213,448

(22) Filed: Jun. 19, 2008

(30) Foreign Application Priority Data
Jun. 22, 2007 (JP) 2007-165451

(51) Int. Cl. C12Q 1/68

(52) U.S. Cl. 435/6

(57) ABSTRACT

It is intended to provide a technique for amplifying, individually and in parallel, nucleic acids contained in a mixture of plural kinds of nucleic acid samples. The present invention provides a nucleic acid analysis method comprising amplification means, whereby amplification reaction is performed in a reaction solution comprising a homogeneous solvent and comprising at least plural template nucleic acids and solid phase carriers comprising one or more kinds of amplification probes immobilized on the surface, to prevent amplified products attributed to two or more template nucleic acids from being replicated in one solid phase carrier. According to the present invention, plural kinds of analyte nucleic acid samples in a mixed state can be amplified individually and in parallel. This method achieves one solid phase carrier-one nucleic acid. Therefore, a higher density of solid phase carriers with obtained amplified products is easily achieved, leading to improved throughput of amplified product analysis. Reactions in all the amplification reaction steps are performed under homogeneous solvent conditions. Therefore, the method of the present invention is performed by convenient procedures and as such, is suitable to automation.

### CLAIM OF PRIORITY

The present application claims priority from Japanese application JP 2007-165451 filed on Jun. 22, 2007, the content of which is hereby incorporated by reference into this application.

### BACKGROUND OF THE INVENTION

1. Field of the Invention

The present invention relates to an analysis method comprising amplifying, individually and in parallel, nucleic acid samples contained in a nucleic acid mixture by use of primers immobilized in advance on solid phase carriers. The present invention also relates to a kit and an apparatus necessary for the individual and parallel amplification and analysis.

2. Background Art

Nucleic acid sequence determination, genetic diagnosis, gene expression analysis, and mutation analysis require amplifying nucleic acids as analytes in advance to an amount sufficient for securing the detection precision of the analysis. One opinion says that nucleic acid amplification does not accurately reflect the sequences or quantitative ratio of the original nucleic acids and presents problems in analysis results. Thus, the development of a technique for directly detecting one molecule without performing amplification (single-molecule measurement) has been pursued energetically. However, this technique still has high hurdles to surmount for its actual practical use. The amplification of analyte nucleic acids is essential for nucleic acid analysis steps at the present time.

On the other hand, analyte nucleic acids are provided in most cases as a mixture of nucleic acids having different kinds of sequences. The nucleic acids contained in the mixture must be amplified individually, even when all the nucleic acids in the mixture are used as analytes or even when only particular nucleic acids in the mixture are used as analytes.

Techniques for amplifying nucleic acids are broadly classified into two groups.

One of them is cloning for biologically amplifying nucleic acids by use of E. coli or the like. The other technique is in vitro amplification by chemical reaction by use of an enzyme (Molecular Cloning: A Laboratory Manual (Third Edition), Cold Spring Harbor Laboratory Press).

In the cloning method, in general, nucleic acid fragments are first inserted into vectors, and the vectors containing the fragment of interest are subsequently introduced into hosts such as E. coli. The hosts usually form colonies on an amplification medium (e.g., an agar plate). Each colony is attributed to each individual host and formed by the host amplified on the order of several millions. Each colony is individually transferred to a container. The host cells can further be replicated and individually amplified in a liquid medium. The target nucleic acids are collected from the amplified host cells and analyzed. In this approach, the target nucleic acids can be isolated at the colony formation stage even from the original mixture of plural kinds of nucleic acids and can be amplified individually by the subsequent replication in a liquid medium. On the other hand, a biological amplification rate of the host cells is a rate-limiting factor in all the steps. Thus, this approach requires an enormous amount of operation time. In addition, the approach presents the problem of complicated procedures which are not suitable to automation and a large scale.

On the other hand, typical examples of the amplification method using an enzyme include PCR (Polymerase Chain Reaction). In this method, short nucleic acids (primers) having sequences complementary to both terminal sequences of a nucleic acid region of interest are prepared in advance. The primers are extended by use of thermostable DNA polymerase. Then, the nucleic acids are denatured under high temperature conditions. The temperature is subsequently lowered, whereby the redundant primers excessively added in advance are complementarily annealed again to the target nucleic acids to cause extension reaction. In this method, extension products of this primer are used as amplified products. Finally, 2ₙ (n represents the number of a procedure of raising and lowering the temperature) nucleic acids can be obtained by repeating the procedure of raising and lowering the temperature. An alternative method is, for example, Rolling Circle Amplification which involves continuously synthesizing a complementary strand of circular DNA as a template by use of bacteriophage-derived DNA polymerase capable of strand displacement. This method can also exponentially amplify circular DNA up to 10⁹ copies. In all of these methods, a dramatically large amount of nucleic acids can be obtained in a short time in a tube. Furthermore, these methods are performed by simple procedures and as such, are suitable for automation. On the other hand, these amplification methods, unlike the cloning method, are unsuitable for individually amplifying a mixture of plural kinds of nucleic acid samples. Specifically, these methods permit simultaneous amplification but cannot isolate different kinds of nucleic acids. PCR may generally require, for example, preparing primer sequences respectively specific for nucleic acids or separating amplified products by electrophoresis, for isolating target nucleic acids from a mixture. In either case, such very complicated procedures are not suitable for analyzing a large amount of samples. Moreover, the means for preparing specific primer sequences can be adopted only for known sequences and is not used for analytes having an unknown sequence.

JP Patent Publication (Kohyo) No. 10-505492A (1998) discloses a nucleic acid amplification technique which overcomes the disadvantages of the cloning and PCR methods and exploits the advantages of these methods. This technique is PCR amplification on a solid phase carrier. This method for detecting the presence of target nucleic acids in a mixture of plural kinds of nucleic acid samples comprises performing PCR reaction on a solid phase carrier comprising amplification primers specific for the target nucleic acids immobilized in advance and determining the presence of the target nucleic acids based on the presence or absence of amplified products. Specifically, target nucleic acid-specific primers necessary for amplifying the nucleic acids of interest are immobilized on a glass substrate or a solid phase equivalent thereto. The surface of the solid phase is covered with a PCR reaction solution, while the primers used in amplification are immobilized thereon. Therefore, amplified products are not leaked into the reaction solution and produced in a form immobilized on the solid phase. The produced products are complementarily annealed to the immobilized primers that exist within the range of the lengths of the products, going into a next amplification step. Finally, amplified products can be obtained in a form where either terminus thereof is immobilized on the solid phase, by repeating this amplification step some dozen times. In this method, target nucleic acids contained in a mixture are isolated and individually amplified, and the presence of the target nucleic acids can be determined based on the presence or absence of amplified products thereof. On the other hand, this method requires designing in advance primers specific for the target nucleic acids and presents the definitive problem of analyte limitations. By contrast, JP Patent Publication (Kohyo) No. 2002-503954A (2002) and Nucleic Acid Research vol. 28 e87 (2000) disclose a PCR amplification method on a solid phase carrier, which solves this problem. This method is different from the above-described technique, in that all nucleic acids contained in a mixture of plural kinds of nucleic acid samples as analytes have a sequence portion capable of forming a complementary strand with common primers used in amplification reaction. Therefore, all the nucleic acids contained in the mixed samples can be amplified by use of common primers immobilized in advance on a solid phase. For the primers immobilized on the solid phase, a very small number of molecules in the mixture of plural kinds of nucleic acid samples are developed on the solid phase surface, whereby the nucleic acid molecules randomly form a complementary strand with the primers immobilized on the solid phase carrier. Complementary strand extension products of the primers are complementarily annealed to their nearest immobilized primers that exist within the range of the lengths of the products, going into a next amplification step. Finally, amplified products can be aggregated within a certain region around the initially produced complementary strand extension products as a center and obtained in a form just as colonies in the cloning method, by repeating this amplification step some dozen times. In this method, each nucleic acid contained in a mixture of plural kinds of nucleic acid samples is individually isolated and provided on the solid phase in a form of colonies of amplified products. Therefore, each nucleic acid in analyte nucleic acid samples provided as a mixture can be analyzed individually. Thus, this method overcomes the disadvantages of the cloning and PCR methods. JP Patent Publication (Kohyo) No. 2002-525125A (2002) discloses a similar method. This method also comprises obtaining colony-like amplified products by use of primers immobilized on a solid phase carrier but is different from the above-described methods (which start from complementary strand extension products), in that nucleic acids to be amplified are immobilized in advance on a solid phase. In all of these approaches, amplified products are commonly obtained as colonies randomly plotted on a solid phase carrier.

An emulsion PCR method has further been reported, which comprises using, as independent reaction vessels, water droplets dispersed in oil to perform PCR amplification reaction (Margulies M., Egholm M., Altman W. E., Rothberg J. M. et al., Nature 437 (7057), 376-80 (2005)). This technique can simultaneously amplify a large number of nucleic acid samples within the water droplets isolated from each other.

### Patent Document 1: JP Patent Publication (Kohyo) No. 10-505492A (1998)

### Patent Document 2: JP Patent Publication (Kohyo) No. 2002-503954A (2002)

### Patent Document 3: JP Patent Publication (Kohyo) No. 2002-525125A (2002)

### Non-Patent Document 1: Molecular Cloning: A Laboratory Manual (Third Edition), Cold Spring Harbor Laboratory Press

### Non-Patent Document 2: Nucleic Acid Research vol. 28 e87 (2000)

Non-Patent Document 3: Margulies M., Egholm M., Altman W. E., Rothberg J. M. et al. Nature 437 (7057), 376-80 (2005)

### SUMMARY OF THE INVENTION

In gene analysis business markets, it is no exaggeration to say that an analysis speed decides the outcome of the business. In analysis steps, the pretreatment of analyte genes, that is, amplification or purification for conducting analysis is most complicated, and a key point is that this step can be performed conveniently in a precise method or a method suitable to automation.

Therefore, it has been demanded to develop a method capable of amplifying, individually and in parallel, a mixture of plural kinds of nucleic acid samples as analytes to smoothly move to the subsequent analysis steps. The above-described PCR amplification reaction using primers immobilized on a solid phase carrier, which are common to analyte nucleic acids, is a very promising approach. However, JP Patent Publication (Kohyo) No. 2002-503954A (2002) is characterized in that amplified product populations (i.e., colonies) of many different nucleic acids are obtained on the surface of a solid phase carrier. In this case, plural colonies are positioned on one solid phase carrier. Amplification primers initially immobilized thereon can be located uniformly on the substrate. However, subsequently added nucleic acids to be amplified are exceedingly difficult to uniformly develop. Colonies may be fused with each other, unless the nucleic acids are developed at a sparse density to some extent. On the other hand, when nucleic acid samples are used at a very sparse density for avoiding such fusion, the surface area of a solid phase carrier must be enlarged with increase in the number of mixed samples. Furthermore, in analysis steps of amplified products of nucleic acids, a low colony density on a solid phase carrier leads to low treatment efficiency. A procedure of, for example, physically cleaving only the solid phases of portions with formed colonies may achieve a higher density of colonies. However, it is actually impossible to cleave, on a colony basis, solid phases containing colonies of allegedly 2 to 3.3 μm² in average size. In JP Patent Publication (Kohyo) No. 2002-525125A (2002), examples of a solid phase carrier other than plane supports typified by glass surface include beads such as latex or dextran beads. However, the distance between colonies is very difficult to control, even when these beads are used as substrates. A higher density of products amplified individually and in parallel on solid phase carrier surface per surface area of the solid phase carrier leads to improved throughput of the subsequent analysis. It has been demanded to develop means for solving this problem.

On the other hand, in the emulsion PCR method, a homogeneous emulsion of a PCR solution in oil is not always easy to prepare. The biggest problem in this method is in that products amplified in the emulsion are not easily collected. For solving this problem, it has been demanded to develop an individual amplification method in a homogenous solution, not in an inhomogeneously distributed reaction solution comprising a mixture of two or more solvents such as oil and liquid phases in an emulsion.

A scheme for solving these problems involves performing sample preparation for achieving one solid phase carrier-one nucleic acid and dispersing the solid phase carriers in a reaction solution for nucleic acid amplification comprising a homogeneous solvent while causing amplification reaction only in the very near neighborhood of the surface of the solid phase carriers. This method achieves one solid phase carrier-one nucleic acid. Therefore, nucleic acids can be amplified individually and in parallel with ease without causing the fusion between colonies, that is, the fusion between amplified products of different kinds of nucleic acids. On the other hand, such sample preparation for achieving one solid phase carrier-one nucleic acid has bad effects. Examples thereof include the problem that a large number of substrates are bound with no nucleic acids. This problem can be solved by detecting the presence or absence of amplified products on the solid phase carrier and separating only the solid phase carrier bound with amplified nucleic acids. Specifically, this can attain the above-described higher density of products amplified individually and in parallel. According to this method, only the solid phase carrier bound with amplified products from a single sample can be used as analytes, leading to high efficiency of analysis procedures. In the conventional method (JP Patent Publication (Kohyo) No. 2002-525125A (2002)), beads are listed as solid phase carriers. Nevertheless, this method is not based on the idea of one bead-one nucleic acid. None of the conventional methods for parallel amplification on a solid phase carrier are designed such that only amplified product areas on a solid phase carrier are provided at a higher density. It is actually impossible to physically cleave a solid phase carrier such that a higher density is achieved. The same object as in the present invention cannot be attained by combining the conventional methods with the separation of amplified areas for a higher density.

Hereinafter, means for achieving the present invention will be described in detail.

One aspect of the present invention relates to a nucleic acid analysis method for simultaneously analyzing plural nucleic acid samples, comprising:

a first step of introducing plural template nucleic acids to plural solid phase carriers such that one solid phase carrier comprising one or more kinds of amplification probes immobilized on the surface is capable of being bound via the probe to a terminal region comprising the 3′ terminus of one template nucleic acid molecule;

a second step of extending the probe with the template nucleic acid as a template to form a first extended probe;

a third step of denaturing the template nucleic acid from the first extended probe;

a fourth step of removing the template nucleic acid;

a fifth step of repeating the steps of (1) annealing a terminal region comprising the 3′ terminus of the extended probe to an unextended probe, (2) extending the unextended probe with the first extended probe as a template to form a second extended probe, and (3) denaturing the first extended probe from the second extended probe, whereby the first and second extended probes are amplified to form a large number of the first and second extended probes on the carrier; and

a sixth step of separating the carrier bound with the first extended probes from the carrier unbound with the first extended probes.

A second aspect of the present invention relates to the nucleic acid analysis method according to the first aspect, further comprising, before the first step, the step of ligating an adaptor having a first sequence to the 3′ termini of the template nucleic acids and ligating an adaptor having a second sequence different from the first sequence to the 5′ termini of the template nucleic acids, wherein each of the plural probes immobilized on the one carrier has a complementary sequence to the first or second sequence.

A third aspect of the present invention relates to the nucleic acid analysis method according to the first aspect, further comprising, before the first step, the step of ligating an adaptor having a first sequence to the 3′ termini of the template nucleic acids and ligating an adaptor having a complementary sequence to the first sequence to the 5′ termini of the template nucleic acids, wherein

each of the plural probes immobilized on the one carrier has a complementary sequence to the first sequence.

A fourth aspect of the present invention relates to the nucleic acid analysis method according to the first aspect, wherein the first to fourth steps are performed in the same container, and the fifth step is performed in different containers individually accommodating each of the plural carriers.

A fifth aspect of the present invention relates to the nucleic acid analysis method according to the first aspect, wherein the first to fifth steps are performed in different containers individually accommodating each of the plural carriers.

A sixth aspect of the present invention relates to the nucleic acid analysis method according to the fourth or fifth aspect, wherein a solution for performing the reaction is common to the different containers individually accommodating each of the plural carriers.

A seventh aspect of the present invention relates to the nucleic acid analysis method according to any of the first to sixth aspects, wherein the fifth step comprises repeating the steps of (1) extending a complementary strand with the first extended probe as a template to form a second extended probe in a bent form such that the complementary strand forms a U shape with its neighboring probe on the same solid phase carrier, and (2) heat denaturing the bent form to form a single-stranded nucleic acid immobilized on the carrier, which is then used as a template in a next cycle.

An eighth aspect of the present invention relates to the nucleic acid analysis method according to any of the first to seventh aspects, wherein in the first to fifth steps, the reaction solution is constantly stirred.

A ninth aspect of the present invention relates to the nucleic acid analysis method according to any of the first to seventh aspects, wherein in the first to fifth steps, the plural carriers are located at a distance longer than the length of the template nucleic acid from each other.

A tenth aspect of the present invention relates to a nucleic acid analysis method for simultaneously analyzing plural nucleic acid samples, comprising:

a first step of introducing plural template nucleic acids to plural solid phase carriers such that one solid phase carrier comprising one kind of probes immobilized on the surface is capable of being bound via the probe to a terminal region comprising the 3′ terminus of one template nucleic acid molecule;

a second step of extending the immobilized probe with the template nucleic acid as a template to form a first extended probe;

a third step of denaturing the template nucleic acid from the first extended probe;

a fourth step of removing the template nucleic acid;

a fifth step of repeating the steps of (1) annealing a terminal region comprising the 3′ terminus of the first extended probe to another kind of suspended probe added to the reaction solution, (2) extending the suspended probe with the first extended probe as a template to form a second extended probe, and (3) denaturing the first extended probe from the second extended probe, whereby the first and second extended probes are amplified to form a large number of the first and second extended probes on the carrier; and a sixth step of separating the carrier bound with the first extended probes from the carrier unbound with the first extended probes.

An eleventh aspect of the present invention relates to the nucleic acid analysis method according to the tenth aspect, wherein the step (3) in the fifth step comprises partially denaturing only the terminal regions of a double-stranded nucleic acid composed of the first and second extended probes to form single-stranded terminal regions, complementarily annealing the single-stranded terminal regions to the immobilized probe or the suspended probe, and performing extension reaction while denaturing the double-stranded portion of the template nucleic acid by use of DNA polymerase capable of strand displacement.

A twelfth aspect of the present invention relates to the nucleic acid analysis method according to the tenth aspect, wherein a substance which has an increased viscosity or is gelled during denaturing (70° C. to 100° C.) and has a decreased viscosity or is in a solution state during complementary annealing (20° C. to 60° C.) is allowed to coexist in the reaction solution, and

after the capturing of the template nucleic acid by the carrier, the nucleic acid amplification reaction is performed in a state where the suspended probes are dispersed in gel.

A thirteenth aspect of the present invention relates to the nucleic acid analysis method according to any of the first to twelfth aspects, wherein the first to fifth steps are performed, during which an anchor sequence for separation which is neither complementary nor identical to the first and second sequences of the adaptors is added to a probe sequence annealed to the 5′ terminus of the template nucleic acid, and wherein, in the sixth step, only the solid phase carrier with obtained amplified products is separated by use of a column bound with a probe complementary to the anchor sequence.

A fourteenth aspect of the present invention relates to the nucleic acid analysis method according to any of the first to twelfth aspects, wherein the first to fourth steps are performed, during which a third sequence which is neither complementary nor identical to the first and second sequences of the adaptors is added to a probe sequence annealed to the 5′ terminus of the template nucleic acid, and wherein, the method further comprises the step of sequencing the template nucleic acid which is not an amplified product by use of a primer having a sequence complementary to the third sequence.

A fifteenth aspect of the present invention relates to the nucleic acid analysis method according to any of the first to fourteenth aspects, wherein only the solid phase carrier with obtained amplified products is separated by adding a double strand-specific intercalator to the amplification reaction solution or to a solid phase carrier suspension after the completion of amplification reaction and detecting/collecting only the solid phase carrier that emits a fluorescence derived from the intercalator from the solution.

A sixteenth aspect of the present invention relates to the nucleic acid analysis method according to any of the first to fifteenth aspects, wherein a reaction solution comprising 10³ or less template nucleic acid molecules for a reaction system using 10⁶ solid phase carriers is prepared to prevent amplified products attributed to two or more template nucleic acids from being replicated on one solid phase carrier.

A seventeenth aspect of the present invention relates to the nucleic acid analysis method according to any of the first to sixteenth aspects, wherein the amplification reaction is performed in a solution comprising a homogeneous solvent.

An eighteenth aspect of the present invention relates to the nucleic acid analysis method according to any of the first to seventeenth aspects, wherein the solid phase carriers are beads.

### BRIEF DESCRIPTION OF THE DRAWINGS

FIG. 1 is a diagram showing one embodiment of reaction steps of the present invention.

FIG. 2 is results of Poisson probability analysis for achieving one solid phase carrier-one nucleic acid.

FIG. 3 is a diagram illustrating the effect of preventing the non-specific adsorption of DNA molecules onto beads.

FIG. 4 is a schematic diagram of a cell for securing the distance between beads.

FIG. 5-1 is a schematic diagram of a cell for securing the distance between beads.

FIG. 5-2 is a schematic diagram of a cell for securing the distance between beads.

FIG. 6-1 is a schematic diagram of the step of introducing a probe for separating beads with obtained amplified products from beads with no obtained amplified products.

FIG. 6-2 is a schematic diagram of the step of introducing a probe for separating beads with obtained amplified products from beads with no obtained amplified products.

FIG. 7 is a schematic diagram of the step of separating beads with obtained amplified products from beads with no obtained amplified products.

FIG. 8 is a schematic diagram of the step of separating beads with obtained amplified products from beads with no obtained amplified products.

FIG. 9-1 is a schematic diagram of a reaction step for performing single-molecule measurement by use of amplified products on beads.

FIG. 9-2 is a schematic diagram of a reaction step for performing single-molecule measurement by use of amplified products on beads.

FIG. 10 is a schematic diagram of the step of introducing a probe for separating beads with obtained amplified products from beads with no obtained amplified products.

FIG. 11-1 is a diagram showing one embodiment of reaction steps of the present invention.

FIG. 11-2 is a diagram showing one embodiment of reaction steps of the present invention.

FIG. 12 is a diagram showing one embodiment of reaction steps of the present invention.

FIG. 13 is a diagram showing results of one embodiment of reaction steps of the present invention.

### DESCRIPTION OF SYMBOLS

101 to 105: double-stranded DNA fragment (DNA sample) of approximately some hundreds bases to 1 kb in base length
106: adaptor A (base sequence wholly or partially complementary to 107)
107: adaptor A (base sequence wholly or partially identical to 113)
108: adaptor B (base sequence wholly or partially complementary to 109)
109: adaptor B (base sequence wholly or partially identical to 112)
111: magnetic bead
112: probe A immobilized on magnetic bead
113: probe B immobilized on magnetic bead
121: complementary strand extension product of probe
122: complementary strand extension product of probe (sequence complementary to 112)
131: new DNA product extended from 112
132: new DNA product extended from 112
401: capturing cell (reaction cell)
402: inlet
403: outlet
405: cover
411: opening
412: bead
501: plane
502: hole
503: cover
504: solution
505: inlet
506: outlet
511: bead
521: aspiration
551: plane
552: magnet
553: magnetic force shield
701: separation/purification column
702: bead after amplification reaction
711: bead with captured product
712: bead with no captured product
713: eluted bead
801: fluorescent bead with captured product
901: magnetic bead
912: probe A immobilized on magnetic bead
913: probe B immobilized on magnetic bead
916: one strand of double-stranded DNA fragment
917: complementary strand to 916
921: adaptor A
922: adaptor A (base sequence wholly or partially identical to 913)
923: adaptor B (base sequence wholly or partially identical to 912)
924: adaptor B (base sequence wholly or partially complementary to 923)
925: adaptor C
926: adaptor C (base sequence wholly or partially complementary to 925)
931: complementary strand extension product of 912
932: complementary strand extension product of 912
933: complementary strand extension product of 912
941: new DNA product extended from 912
942: new DNA product extended from 912
201: Sepharose bead
202: direction of extension of 212
212: probe A immobilized on bead
213: one strand of double-stranded DNA fragment of approximately some hundreds bases to 1 kb in base length
214: complementary strand of 213
215: base sequence constituting adaptor A (base sequence wholly or partially complementary to 216)
216: base sequence constituting adaptor A
231: initial complementary strand extension product
232: initial complementary strand extension product
235: extension product having sequence complementary to 232
236: extension product having sequence complementary to 231
237: extension product having sequence complementary to 212
241: direction of extension reaction while denaturing double-stranded complementarily annealed region as a template
242: direction of extension reaction while denaturing double-stranded complementarily annealed region as a template
243: partial denatured portion
244: partial denatured portion
245: denatured portion in the other terminal region
246: denatured portion in the other terminal region
250: first complementary strand extension product
251: extension product having sequence complementary to 250
255: denatured portion in the other terminal region
256: denatured portion in terminal region

### DETAILED DESCRIPTION OF THE PREFERRED EMBODIMENTS

### 1. First Method

Examples of a first method of the present invention includes an embodiment wherein the 5′ termini of primers used in PCR are immobilized on solid phase carrier surface, and PCR is performed by dropwise addition onto the this solid phase carrier or on the solid phase dipped in a liquid phase (hereinafter, the primer immobilized on the solid phase is referred to as a probe).

### 1.1 Probe

Preferably, two kinds of probes are used. However, one kind or two or more kinds of probes may be used. Examples of a method for immobilizing probes onto solid phase carriers include, but not particularly limited to, covalent bond, ionic bond, physical adsorption, and biological binding (e.g., biotin-avidin or streptavidin-avidin binding or antigen-antibody binding).

### 1.2 Solid Phase Carrier

It is here assumed that magnetic bead particles of approximately 1 to 100 μm in particle size are used as solid phase carriers for immobilization. Materials for the solid phase carriers are insoluble in water, and examples thereof include, but not particularly limited to; metals such as gold, silver, copper, aluminum, platinum, titanium, and nickel, alloys such as stainless and duralumin, silicon, glass materials such as, glass, quartz glass, and ceramics, plastics such as polyester resins, polystyrene, polypropylene resins, nylon, epoxy resins, and vinyl chloride resins, agarose, dextran, cellulose, polyvinyl alcohol, and chitosan. Likewise, the shapes of the carriers are not particularly limited.

### 1.3 Preparation of Analyte Samples

Nucleic acid samples serving as analytes are fragmented in advance by restriction enzyme cleavage or ultrasonic cleavage, when individual sample base lengths are long, as with genomic samples (e.g., 1000 to 2000 or more bases). Another possible cleavage means involves, for example, cleaving nucleic acid samples by moving a solution containing the nucleic acid samples up and down in a very thin needle. Any physical cleavage means may be used. This cleavage step can be omitted for a mixed sample composed of a fragment group of some thousands of bases in maximum length, such as cDNA or RNA samples. It is here assumed that DNA fragments cleaved by ultrasonic cleavage are used as nucleic acid samples.

### 1.4 Ligation of Adaptor to Analyte Sample

When one kind of probes are immobilized on the solid phase carriers, a site (adaptor) having a sequence complementary to the probe and a site having a sequence identical to the probe are subsequently ligated to both termini of the DNA samples. When two kinds of probes are immobilized thereon, a site (adaptor) having a sequence complementary to one of the probes and an adaptor having a sequence identical to the other probe are ligated to both termini of the DNA samples. When two or more kinds of probes are used, an adaptor having a sequence complementary to any of the probes and an adaptor having a sequence identical to any of the probes may be ligated to both termini of the DNA samples.

### 1.5 PCR Reaction

Probe-immobilized beads are prepared in advance in a microtube in an amount capable of binding in a one-to-one relationship to the analyte DNA molecules ligated with the adaptors. To this microtube, a PCR reaction solution (reaction buffer, dNTP mixture, magnesium solution) is added, and the analyte DNAs are further added. Finally, thermostable DNA polymerase is added dropwise thereto, and the microtube is set in a thermal cycler. Reaction is first performed at approximately 95° C. to completely heat denature the DNAs into single strands. Then, the temperature is lowered to approximately 55° C., at which the adaptor portion of the DNA is complementarily annealed to the immobilized probe on the bead. Subsequently, the temperature is raised to 72° C., at which probe extension reaction is performed with the complementarily annealed DNA as a template. Then, the whole reaction solution is temporarily removed. A solution having denaturing effects, such as an alkaline solution, is further added to the microtube, or the addition of formamide or the like also having denaturing effects is combined with heating effects, whereby the DNA used as a template is denatured from complementary strand extension products of the probe. Then, this solution is completely removed.

Then, the microtube is further washed, if necessary, with a 10 mM Tris buffer or 1×TE (10 mM Tris, 1 mM EDTA (ethylenediaminetetraacetic acid)) buffer, and the solution is completely removed. In this step, the DNA serving as a template in the complementary strand extension of the probe and the redundant initially added DNA that has not participated in complementary annealing are all removed. As a result, the complementary annealing between other beads and DNAs and the non-specific adsorption of DNAs to beads can be prevented in subsequent reactions. This washing/removal step is very important for achieving one solid phase carrier-one nucleic acid. After the washing treatment, a PCR reaction solution is added again to the microtube. DNA polymerase is added dropwise thereto, and the microtube is set in a thermal cycler. Subsequently, thermal cycle reaction (30 to 50 cycles each involving 94° C. for 30 sec.→55° C. for 30 sec.→72° C. for 60 sec.) is started. In this step, only the complementary strand extension products of the probe immobilized on the bead in the initial step function as templates. The terminal region of this complementary strand extension product forms again a complementary strand with the probe immobilized on the bead to extend the probe, whereby a new DNA strand (complementary strand extension product of the probe) is formed on the bead. In this procedure, the DNA as a template on the solid phase must form a complementary strand in a bent form such that the complementary strand forms a U shape with its neighboring probe on the same bead. If this DNA forms a complementary strand with a probe immobilized on other adjacent beads, one solid phase carrier-one nucleic acid, that is, individual amplification cannot be achieved. Therefore, the beads must be located in advance in a distance longer than the length of the analyte DNA from each other to perform reaction.

### 1.6 Method for Securing Distance Between Beads

To secure the distance between the beads, the reaction solution may be stirred constantly, or a plate-like cell (microcell) in which openings having a size capable of capturing each of the beads are formed in advance may be used. In this case, one bead is placed in advance in one opening in the cell, and a reaction solution charged around this opening and the bead. A sufficient distance between the beads can be secured. As a result, the crossover of extension reaction between the beads is completely prevented. Possible means for locating beads involves: forming openings for capturing the beads; developing beads onto a plane in which plural holes smaller in size than the beads are formed and capturing the bead onto the upper portion of the holes by aspiration from below the holes; or using magnets in a pin form located below a plane substrate to locate magnetic beads on the pins.

The DNA thus amplified is one kind of DNA for one bead. The analytes starting from a mixture can be amplified individually by each of the beads.

### 2. Second Method

Examples of a second method of the present invention include an embodiment wherein one kind of probes are immobilized on bead surface, and amplification is performed in combination with another kind of suspended probes (non-immobilized primers diffused in the liquid phase of a reaction solution). This method is characterized in that only the terminal regions of an extended complementary strand are partially denatured without completely denaturing the double-stranded portion thereof. A temperature of 90° C. or higher is required for completely denaturing the double-stranded sequence of an extended complementary strand. However, only the terminal regions which are easily denatured can be denatured partially into single strands under temperature conditions of approximately 60 to 80° C.

The terminal denatured portion of this partially denatured complementary strand that is nearer to the bead surface is complementarily annealed to the probe immobilized on the solid phase surface, whereas the other terminus is complementarily annealed to the non-immobilized primer diffused in the solution. Then, extension reaction is allowed to proceed, while the complementary annealing of the double-stranded portion of the double-stranded DNA as a template having the partially denatured termini is denatured by use of DNA polymerase capable of strand displacement (e.g., RepliPHI™ Phi29 DNA Polymerase (100 units/μL) (EPICENTRE)). Reaction steps under temperature conditions for partial denaturing and under temperature conditions for complementary annealing and extension reaction can be repeated plural times to obtain amplified products on the bead.

In this method, denaturing is not performed, unlike usual PCR, under high temperature conditions of 90° C. or higher. Therefore, any portion of the double strand is consistently complementarily annealed with the strand immobilized on the bead. As a result, extended complementary strands obtained from the primers diffused in the liquid phase are neither separated from the bead surface nor diffused. Unlike the first method, one of the primers used in amplification reaction is non-immobilized and has a much higher degree of freedom than that of the immobilized primers. Therefore, this second method is characterized in that amplification efficiency is much higher than that obtained using only the immobilized primers.

### 3. Third Method

Examples of a third method of the present invention include an embodiment wherein a medium which has a viscosity increased under high temperature conditions required for denaturing DNAs is added to a solution. As in the second method, one of amplification primers is immobilized on a bead, and the other primer is non-immobilized and diffused in a reaction solution. In this state, reaction is performed. Both the methods can be expected to have higher amplification efficiency than that obtained using only the immobilized primers. On the other hand, an extended complementary strand of the non-immobilized primer must be prevented from moving from the neighborhood of the bead surface in a state where its double-stranded state is completely denatured under high temperature conditions of 90° C. or higher.

Thus, a solvent (e.g., Mebiol Gel™ (Mebiol Inc.) or methylcellulose gel) which has a viscosity increased under high temperature conditions (70 to 100° C.) is added to a reaction solution. An extended strand obtained from the non-immobilized primer cannot freely move under high temperature conditions which provide complete denaturing. On the other hand, the viscosity of the reaction solution is decreased under temperature conditions (20 to 60° C.) which form complementary annealing. In this case, the non-immobilized primers diffused in the solution can freely move. Therefore, complementary strand extension reaction smoothly proceeds on the bead surface.

According to such some schemes for preventing the DNA extension products produced by complementary annealing from being separated from the neighborhood of the bead surface, DNA strands can be amplified individually in the homogeneous solution and collected easily.

### 4. Separation of Bead as Analyte

Subsequently, only the bead as an analyte bound with amplified products is separated. The throughput of the subsequent analysis step can be improved by removing the bead with no amplified products. Possible separation means involves adding an anchor sequence for separation to a probe sequence annealed to the terminus of the amplified product different from the terminus immobilized on the bead and using a column bound with a probe complementary to this anchor sequence. The beads after amplification reaction are added to the column. As a result, the bead with amplified products is trapped by the probe on the column, whereas the bead with no amplified products passes through the column without being trapped. Subsequently, a solution with a low salt concentration is added to the column to denature the complementary annealing between the amplified product on the bead and the probe on the column. These beads may be eluted into the solution and collected.

Alternative possible means involves using the double-stranded forms of the amplified products. This means involves adding a double strand-specific intercalator to the amplification reaction solution or to a bead suspension after the completion of amplification reaction. From this solution, only the bead that emits a certain fluorescence is detected and collected by use of, for example, a flow cytometer, whereby only the bead with amplified products can be collected easily.

Hereinafter, the present invention will be described with reference to Examples.

### EXAMPLES

### Example 1

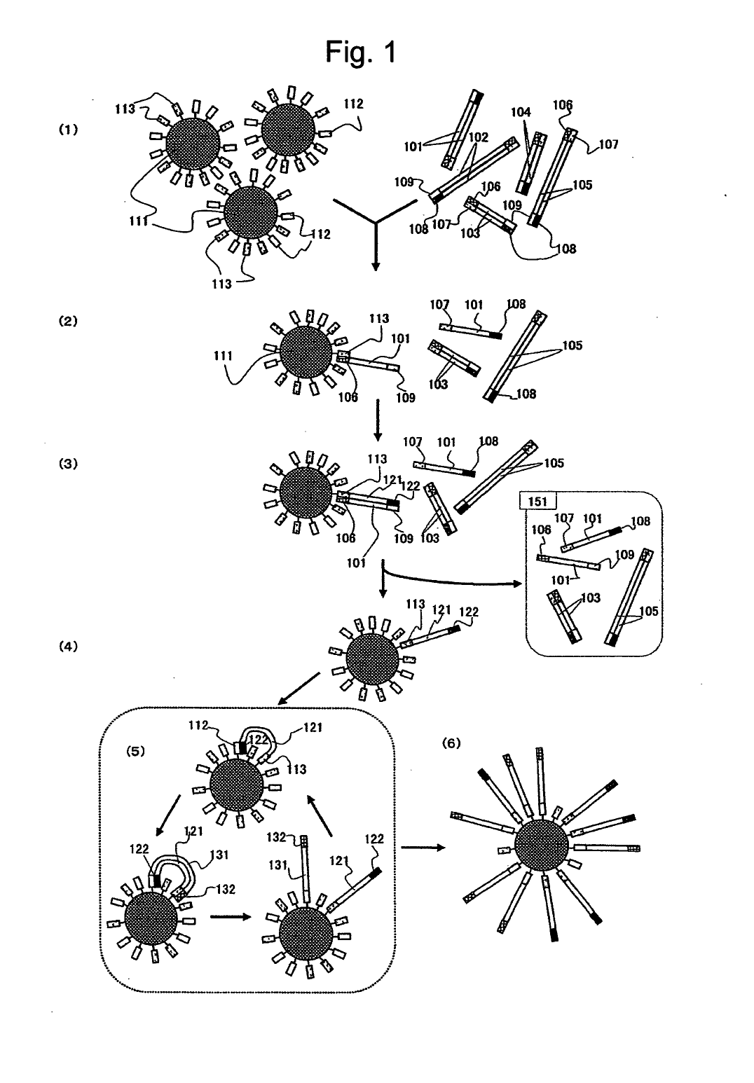

The steps of the present Example are schematically shown in FIG. 1. Magnetic beads (2.8 μm in diameter; Dynal BIOTECH) activated with carboxylic acid groups were used as solid phase carriers. 50 μL (1×10⁸ beads) of a solution containing the carboxylic acid group-activated magnetic beads well suspended in advance was measured into a 2.0 mL microtube. Magnets were placed on the side wall of the tube, and the supernatant was removed with the magnetic beads captured. To wash the beads, 100 μL of a MES Buffer (25 mM MES (2-Morpholinoethanesulfonic acid) (pH 6.0), 0.1% (w/v) Tween 20) was added thereto, and the mixture was stirred and shaken at room temperature for 10 minutes. Then, the supernatant was removed. This step was repeated again, and the supernatant was removed. Subsequently, 30 μL each of solutions of probes A and B diluted to 2.5 pmol/μL with a MES buffer was added thereto, and the mixture was stirred and shaken at room temperature for 30 minutes. The probes A and B comprise an amino group inserted into the 5′ terminus thereof and hexaethylene glycol of 18 atoms as a spacer inserted between the amino group and the probe base sequence. The length of the spacer is not particularly limited. Alternatively, usual bases (e.g., a poly-T sequence) may be used as a spacer. Then, 30 μL of an EDC (1-Ethyl-3-(3-dimethylaminopropyl)carbodiimide) solution (adjusted to 0.1 mg/μL with a MES Buffer) and 10 μL of a MES buffer were added thereto, and the mixture was stirred and shaken overnight under conditions of 4° C. to immobilize the probes onto the magnetic beads. After the completion of reaction, the supernatant was removed. The carboxylic acid groups on the bead unbound with the probe were blocked. Specifically, 200 μL of a Blocking Buffer (50 mM Tris (pH 7.5), 0.1% (w/v) Tween 20) was added thereto, and the mixture was stirred and shaken at room temperature for 15 minutes. Then, the supernatant was removed. This step was repeated 4 times. Then, 200 μL of a 10 mM Tris (pH 7.5)-0.1% (w/v) Tween 20 solution was added thereto to obtain probe-immobilized beads at a final concentration of 5×10⁵ beads/mL. In FIG. 1, reference numeral 111 denotes a magnetic bead; reference numeral 112 denotes a probe A immobilized on the magnetic bead; and reference numeral 113 denotes a probe B immobilized on the magnetic bead.

Subsequently, analyte DNA samples were prepared. The DNA samples are a mixture of double-stranded DNA fragments 101 to 105 of approximately some hundreds bases to 1 kb in base length. An adaptor A having sequences 106 and 107 and an adaptor B having sequences 108 and 109 were ligated in advance to both termini of the double-stranded DNA fragments 101 to 105. The base sequence 106 constituting the adaptor A is wholly or partially complementary to the base sequence 107. The base sequence 107 is wholly or partially identical to the probe B 113 immobilized on the magnetic bead 111. The base sequence 108 constituting the adaptor B is wholly or partially complementary to the base sequence 109. The base sequence 109 is wholly or partially identical to the probe A 112 immobilized on the magnetic bead 111.

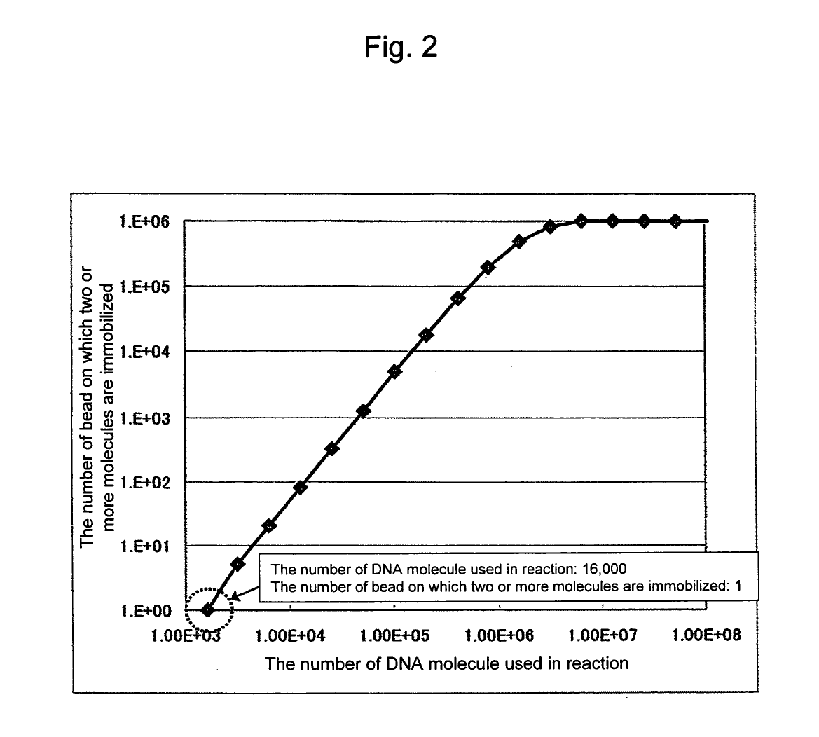

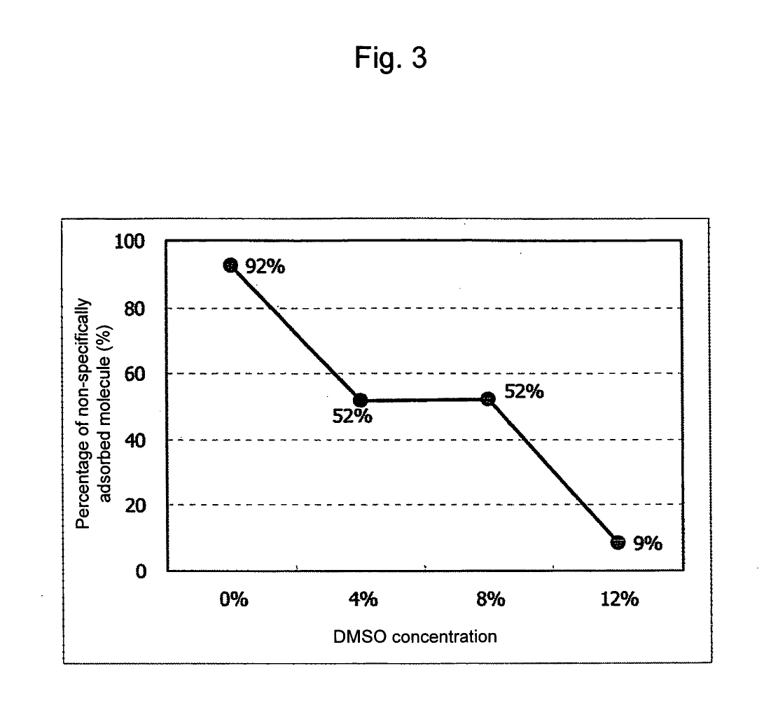

Subsequently, the probe A (112)- and the probe B (113)-immobilized magnetic beads 111 were mixed with the double-stranded DNA fragments ligated with the adaptor A (106 and 107) and the adaptor B (108 and 109) to complementarily anneal the probes on the magnetic beads and the DNA fragments. An important thing here is a mixing ratio between the beads and the DNA fragments. When the numbers of a bead and a DNA molecule (single-stranded DNA is counted as one molecule, and double-stranded DNA is counted as two molecules) used in reaction are defined as N and n, respectively, the average value (λ) of the number of a DNA molecule bound per bead is indicated in n/N. A requirement for binding reaction conditions is that this value does not exceed 1. When this reaction condition is calculated from Poisson probability, a probability (P) that two or more DNA molecules are bound with one bead can be indicated in P=1−(1+λ)e^{-λ}. A probability that two or more DNA molecules are immobilized per bead was plotted in a reaction system using 10⁶ beads (FIG. 2). This plot demonstrated that to achieve one bead-one DNA (one solid phase carrier-one nucleic acid), a reaction solution containing 10³ or less DNA molecules must be prepared, whereby the number of a bead comprising two or more molecules immobilized thereon can be one or less in a reaction system using 10⁶ beads. Thus, in the present Example, 5×10² molecules of double-stranded DNA fragments were mixed for 10⁶ beads to perform reaction (FIG. 1(1)). The mixed solution of the beads and the DNA fragments was mixed with 4 μL of a 10×PCR buffer (600 mM Tris-SO₄ (pH 8.9), 180 mM Ammonium Sulfate), 1.6 μL of 50 mM MgSO₄, 0.8 μL of a 10 mM dNTP Mix (mixed solution of dATP, dCTP, dGTP, and dTTP), and 0.4 μL of Platinum Taq DNA Polymerase High Fidelity (Invitrogen) (5 units/μL), and the total amount of the solution was adjusted to 40 μL with distilled water (DW) to prepare a reaction solution. Subsequently, the DNA fragments were completely denatured into single strands at 94° C. for 60 seconds. Subsequently, complementary strand formation and extension reaction were performed by incubation at 50° C. for 120 seconds→at 72° C. for 120 seconds. In this step, the adaptor 106 ligated to the terminus of the DNA molecule (composed of 106, 101, and 109) was complementarily annealed to the probe B 113 on the bead such that the probe B 113 was extended with the DNA molecules 101 and 109 as templates (FIGS. 1(2) and 1(3)). In FIG. 1, reference numerals 121 and 122 denote a complementary strand extension product of the probe. The terminus of the extension product was ligated with the portion 122 having a sequence complementary to another kind of the probe A 112 immobilized on the bead. An important thing for this complementary strand formation and extension reaction is that extension reaction proceeds after the complementary strand formation between the DNA molecule and the probe. When the DNA molecule exists on the bead by non-specific adsorption, undesired products might be produced. When the DNA molecules take a higher order structure, such reaction on solid phase carriers such as beads might significantly reduce complementary strand formation efficiency with the probe on the bead. Thus, a Denhardt's solution (composition of 50×Denhardt's solution:1% bovine serum albumin (BSA), 1% Ficoll, 1% polyvinylpyrrolidone) as a non-specific adsorption inhibitor or DMSO (dimethyl sulfoxide) as a denaturant was examined for its influence on non-specific adsorption and complementary strand formation efficiency. As a result, it could be confirmed that the addition of these reagents has the effect of preventing non-specific adsorption (FIG. 3). In addition to these reagents, a polymer compound such as PEG (polyethylene glycol) as an adsorption inhibitor or formamide as a denaturant is effectively added as an additive for supporting the prevention of non-specific adsorption or the enhancement of complementary strand formation efficiency. In the present Example, 0.1% (final concentration) BSA or 8% (final concentration) DMSO was added thereto to perform reaction.

Subsequently, to remove the single-stranded DNA sample (composed of 106, 101, and 109) used as a template in complementary annealing/complementary strand extension, the DNA sample adsorbed on the bead surface, and the redundant DNA sample that had not participated in binding, the microtube was washed with solutions of (i) 0.5 N NaOH (room temperature, 1 min.×two times), (ii) 1×TE (94° C., 1 min.×one time), and (iii) 10 mM Tris (pH 7.5) (94° C., 1 min.×one time). The supernatant was removed (In FIG. 1, DNA samples boxed within a broken line 151). The washing solutions may be supplemented, if necessary, with Tween 20 at a final concentration of approximately 0.01 to 0.1% (w/v) to prevent bead aggregation or adsorption onto the internal wall of a pipette chip. This washing/removal step is essential for achieving one bead-one DNA. Without this step, the residual DNA sample forms second complementary strand extension products with the probe on the bead already having complementary strand extension products in the subsequent amplification step, resulting in amplified products of one bead-plural kinds of DNAs.

Subsequently, the complementary strand extension products on the bead after washing were amplified. The washed beads (10⁶) were mixed with 4 μL of a 10×PCR buffer (600 mM Tris-SO₄ (pH 8.9), 180 mM Ammonium Sulfate), 1.6 μL of 50 mM MgSO₄, 0.8 μL of a 10 mM dNTP Mix, and 0.4 μL of Platinum Taq DNA Polymerase High Fidelity (Invitrogen) (5 units/μL), and the total amount of the solution was adjusted to 40 μL with DW to prepare a reaction solution. The beads were suspended in this solution. 50 cycles each involving 94° C. for 30 seconds (heat denaturing of DNA into single strands)→55° C. for 120 seconds (complementary annealing to the probe immobilized on the bead)→72° C. for 45 seconds (complementary strand extension reaction of the probe) were performed (FIG. 1(5)). Finally, the temperature was set to 72° C. for 10 minutes and then lowered to room temperature. In this amplification step, the complementary strand extension products (121 and 122) of the probe immobilized on the bead in the initial step function as templates. The terminal region 122 of this complementary strand extension product (121 and 122) formed a complementary strand with the probe A 112 immobilized on the bead to extend the probe A 112, whereby a new DNA strand (complementary strand extension products 131 and 132 of the probe) was formed on the bead. In this procedure, the DNA as a template on the solid phase forms a complementary strand in a bent form such that the complementary strand forms a U shape with its neighboring probe on the same bead. The extension product also takes a bent form. In the step of heat denaturing into single strands, this bent form is heat denatured to form single-stranded DNA immobilized on the bead, which is then used as a template in a next cycle. Finally, a large number of amplified products are obtained on the bead (FIG. 1(6)).

In this amplification step, the beads in the reaction solution must be located in a distance longer than the length of the extended DNA strand from each other. This is because when the beads are located close to each other such that the distance between them is shorter than the length of the extension product, the terminus of the extension product different from the terminus immobilized on the bead forms a complementary strand with the immobilized probe on the bead different therefrom to produce DNA extension products between plural beads, which are contradictory to one-bead-one DNA. In the present Example, the distance between the beads was secured by adjusting the concentration of DNA molecules per bead and performing reaction under conditions where the beads are consistently diffused by constantly stirring the reaction solution. A method for this stirring may be stirring in a horizontal direction or rotational stirring in a perpendicular direction using a rotator. Reaction performed under conditions where the uniform suspension of the beads is consistently kept in the reaction solution is essential for this step. As in the above-described step of introducing DNA samples onto beads (production of complementary strand extension products), the prevention of non-specific adsorption of amplified products onto beads and the denaturing of a higher order structure are effective for this step. Therefore, in the present Example, 0.1% (final concentration) BSA or 8% (final concentration) DMSO were added to the reaction solution during amplification reaction, and thermal cycle reaction was performed with horizontal stirring. DNA amplified products starting from one kind of DNA sample were obtained on the bead by these steps. The number of a DNA molecule on the bead was quantified by real-time PCR. In this real-time PCR measurement, ABI7900HT (Applied Biosystems) was used, and reaction and measurement procedures were performed according to the method recommended by Applied Biosystems. As a result of quantification of the number of a DNA molecule on the bead during the production of complementary strand extension products and after amplification reaction, amplified products of complementary strand extension products were confirmed to be produced on the bead. In the real-time measurement, probe-non-immobilized bead samples were prepared as negative controls and subjected to reaction under the same conditions as above. Signals detected from the probe-non-immobilized beads are probably caused by DNA molecules non-specifically adsorbed onto the beads. As a result of comparison with the controls, the values in the real-time PCR measurement results could be confirmed to be surely obtained from the amplified products specifically bound onto the bead.

### Example 2

Magnetic beads (2.8 μm in diameter; Dynal BIOTECH) activated with carboxylic acid groups were used as solid phase carriers. 50 μL (1×10⁸ beads) of a solution containing the carboxylic acid group-activated magnetic beads well suspended in advance was measured into a 2.0 mL microtube. Magnets were placed on the side wall of the tube, and the supernatant was removed with the magnetic beads captured. To wash the beads, 100 μL of a MES Buffer (25 mM MES (pH 6.0), 0.1% (w/v) Tween 20) was added thereto, and the mixture was stirred and shaken at room temperature for 10 minutes. Then, the supernatant was removed. This step was repeated again, and the supernatant was removed. Subsequently, 30 μL each of solutions of probes A and B diluted to 2.5 μpmol/μL with a MES buffer was added thereto, and the mixture was stirred and shaken at room temperature for 30 minutes. Then, 30 μL of an EDC solution (adjusted to 0.1 mg/μL with a MES Buffer) and 10 μL of a MES buffer were added thereto, and the mixture was stirred and shaken-overnight under conditions of 4° C. to immobilize the probes onto the magnetic beads. After the completion of reaction, the supernatant was removed. The carboxylic acid groups unbound with the probe were blocked. Specifically, 200 μL of a Blocking Buffer (50 mM Tris (pH 7.5), 0.1% (w/v) Tween 20) was added thereto, and the mixture was stirred and shaken at room temperature for 15 minutes. Then, the supernatant was removed. This step was repeated 4 times. Then, 200 μL of a 10 mM Tris (pH 7.5)-0.1% (w/v) Tween 20 solution was added thereto to obtain probe-immobilized beads at a final concentration of 5×10⁵ beads/μL.

Subsequently, analyte DNA samples were prepared. The DNA samples are a mixture of double-stranded DNA fragments 101 to 105 of approximately some hundreds bases to 1 kb in base length. As shown in FIG. 1, an adaptor A having sequences 106 and 107 and an adaptor B having sequences 108 and 109 were ligated in advance to both termini of the double-stranded DNA fragments 101 to 105. The base sequence 106 constituting the adaptor A is wholly or partially complementary to the base sequence 107. The base sequence 107 is wholly or partially identical to the probe B 113 immobilized on the magnetic bead 111. The base sequence 108 constituting the adaptor B is wholly or partially complementary to the base sequence 109. The base sequence 109 is wholly or partially identical to the probe A 112 immobilized on the magnetic bead 111.

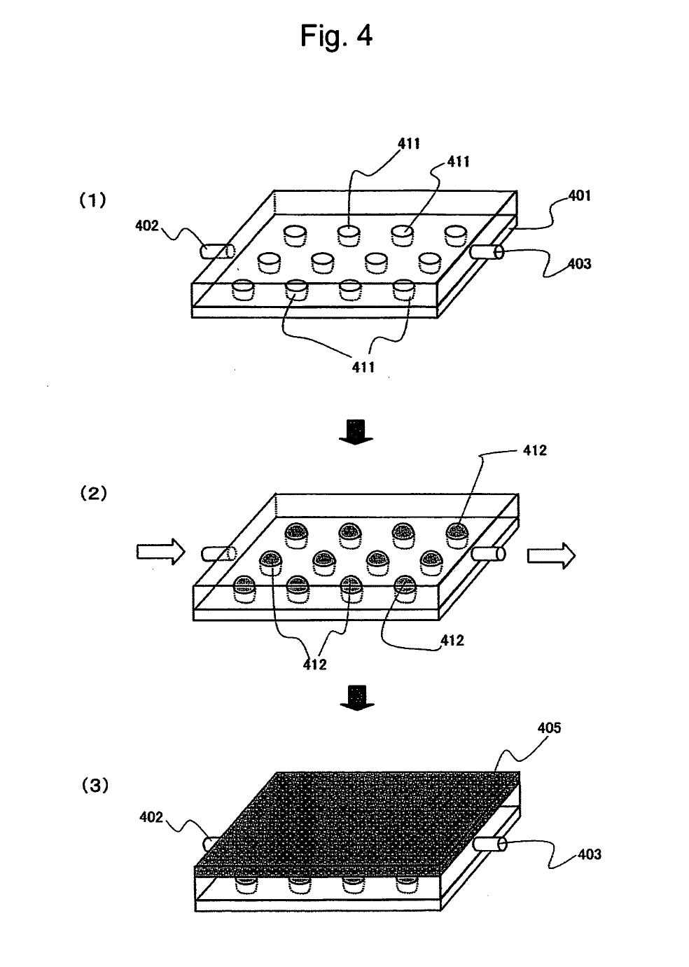

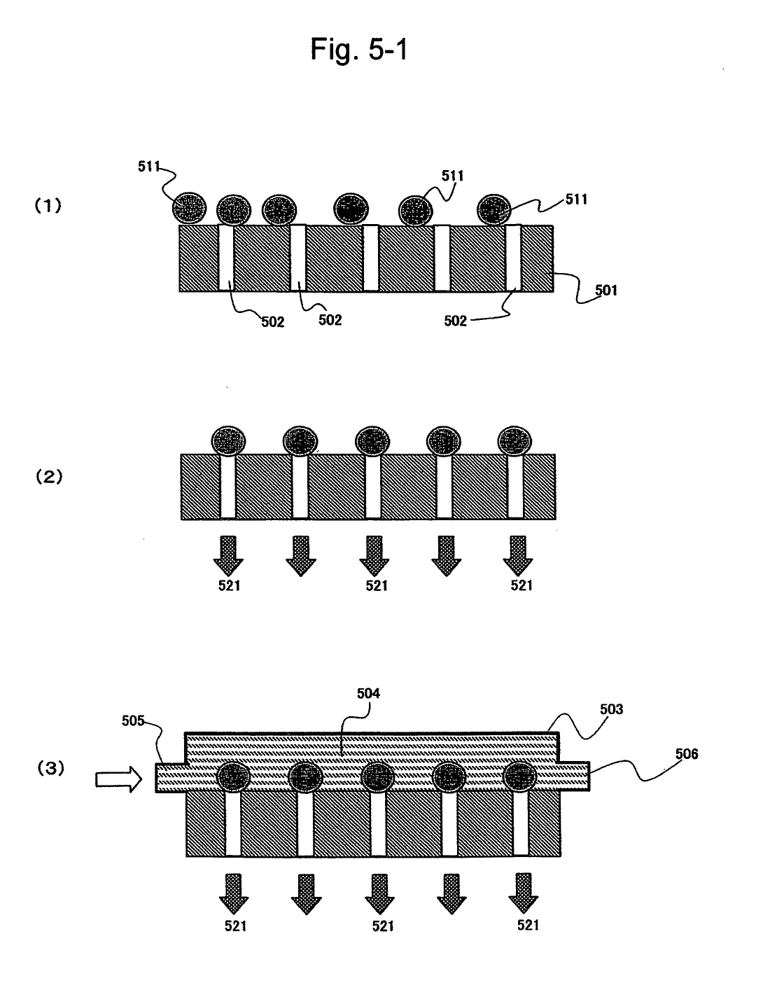

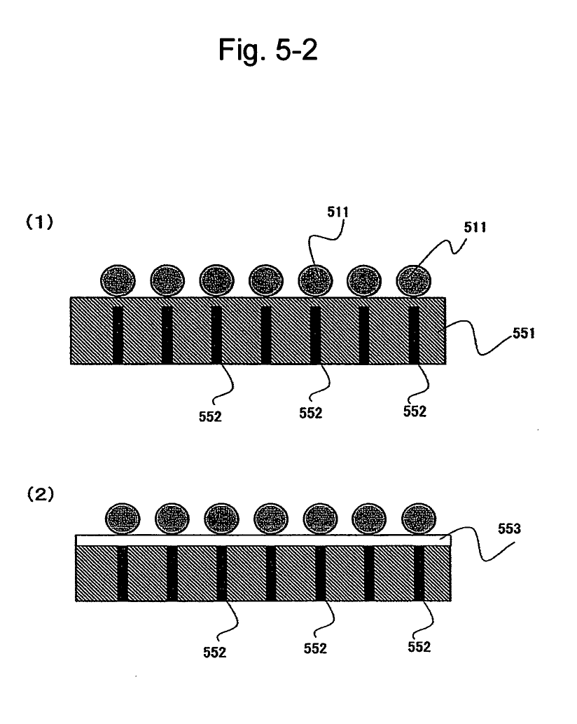

Subsequently, the probe A (112)- and the probe B (113)-immobilized magnetic beads 111 were mixed with the double-stranded DNA fragments ligated with the adaptor A (106 and 107) and the adaptor B (108 and 109) to complementarily anneal the probes on the magnetic beads and the DNA fragments. In the reaction, to secure the distance between the beads longer than the length of the extended DNA strand, the beads were placed in advance on a specialized reaction cell for bead capturing to perform reaction below. The constitution of the reaction cell is shown in FIG. 4. A cell 401 is provided with openings 411 having a size (opening size of approximately 3 to 3.5 μm for the beads of 2.8 μm used in the present Example) capable of holding beads 412 such that the number of openings 411 corresponds with the number of beads 412. These openings are very fine. Therefore, an enormous number of these openings can be placed in an exceedingly narrow area. Specifically, the base length of the analyte DNA samples is, for example, approximately 0.03 μm for 300 bases or approximately 0.1 μm for 1000 bases, since the distance between the bases is approximately 3.5 Å. Therefore, for example, when openings of 3.5 μm in diameter are placed at 1-μm intervals, approximately 6×10⁸ openings can be placed in an area within 1 cm around. The bead suspension is injected from an inlet 402 onto the capturing cell 401 shown in FIG. 4. A solution discharged from an outlet 403 on the opposite side is reinjected into the cell 401. The beads are placed in the openings 411 by such a procedure of injecting and discharging the solution (FIG. 4(2)). Then, the reaction cell 401 is covered with a cover 405 to prevent the leakage of the beads 412. In this case, the beads cannot come out of the openings due to the cover. By contrast, the solution injected from the inlet 402 can freely move around the beads (also around the openings). The unnecessary solution can be removed from the outlet 403. The use of such a reaction cell 401 permitted the supply of a reaction or washing solution onto bead surface with the distance between the beads secured. The shape of the cell may be the shape shown in FIG. 4 or a shape shown in FIG. 5. Specifically, through-holes 502 provided in a plane 501, not the openings, hold beads. The holes are smaller in size than beads 511. The beads can be held on the holes by aspiration 521 from below the holes, whereas the beads can freely move without aspiration. The beads can freely move on the plane without aspiration (FIG. 5-1(1)) but are immobilized on the holes by aspiration from below the holes (FIG. 5-1(2)). This plane is covered with a cover 503 capable of holding a solution, whereby a solution 504 can be held around the beads (FIG. 5-1(3)). The cover 503 is provided with an inlet 505 and an outlet 506 for the solution. The beads are held by aspiration during reaction solution injection/discharge, during reaction, or during washing. After the completion of amplification reaction, the beads can be collected easily by stopping aspiration. Alternatively, beads 511, as shown in FIG. 5-2, can be placed and held on a plane 551 by use of magnets 552 placed in a pin form in the plane 551. In this case, the beads can be controlled such that the beads are captured or released by inserting or removing a magnetic force shield 553 between the beads 511 and the magnets 522. The same effects as in beads capturing by the aspiration are obtained.

To the reaction cell, a solution containing 5×10² molecules of double-stranded DNA fragments for 10⁶ beads, a mixed reaction solution (1×PCR buffer (600 mM Tris-SO₄ (pH 8.9)), 18 mM Ammonium Sulfate), 2 mM MgSO₄, 0.2 mM dNTP, and Platinum Taq DNA Polymerase High Fidelity (0.05 units/μL) were injected to sufficiently fill the reaction cell with the solution. Subsequently, incubation at 94° C. for 60 seconds→at 50° C. for 120 seconds→at 72° C. for 120 seconds was performed. To achieve the prevention of non-specific adsorption of the DNA samples onto the beads and the denaturing of a higher order structure, 0.1% (final concentration) BSA or 8% (final concentration) DMSO was added to the reaction solution to perform the reaction. Subsequently, to remove the single-stranded DNA sample used as a template in complementary annealing/complementary strand extension, the DNA sample adsorbed on the bead surface, and the redundant DNA sample that had not participated in binding, the cell was washed by a flow of solutions of (i) 0.5 N NaOH (1 min.×two times), (ii) 1×TE (1 min.×one time), and (iii) 10 mM Tris (pH 7.5) (1 min.×one time). Finally, the solution was completely removed.

Subsequently, the complementary strand extension products on the bead after washing were amplified. To the washed beads (10⁶), a mixed reaction solution (1×PCR buffer (600 mM Tris-SO₄ (pH 8.9)), 18 mM Ammonium Sulfate), 2 mM MgSO₄, 0.2 mM dNTP, and Platinum Taq DNA Polymerase High Fidelity (0.05 units/μL) were injected to sufficiently fill the reaction cell with the solution. Subsequently, 50 cycles each involving 94° C. for 30 seconds→55° C. for 120 seconds→72° C. for 45 seconds were performed. Finally, the temperature was set to 72° C. for 10 minutes and then lowered to room temperature. To achieve the prevention of non-specific adsorption of the DNA samples onto the beads and the denaturing of higher order structure, 0.1% (final concentration) BSA or 8% (final concentration) DMSO was added to the reaction solution to perform the reaction. DNA amplified products starting from one kind of DNA sample were obtained on the bead by these steps.

### Example 3

An important thing in the present invention is a mixing ratio between the beads and the DNA fragments. To achieve one bead-one nucleic acid, a reaction solution containing 10³ or less DNA molecules must be prepared according to Poisson probability shown in FIG. 2, whereby the number of a bead comprising two or more molecules immobilized thereon can be one or less in a reaction system using 10⁶ beads. According to this calculation, 998,400 beads corresponding to 98.4% of the whole are bound with no DNA fragments. The throughput of the amplified product analysis step can be improved dramatically by separating only the bead bound with the DNA fragment, from which products were obtained at the subsequent amplification step.

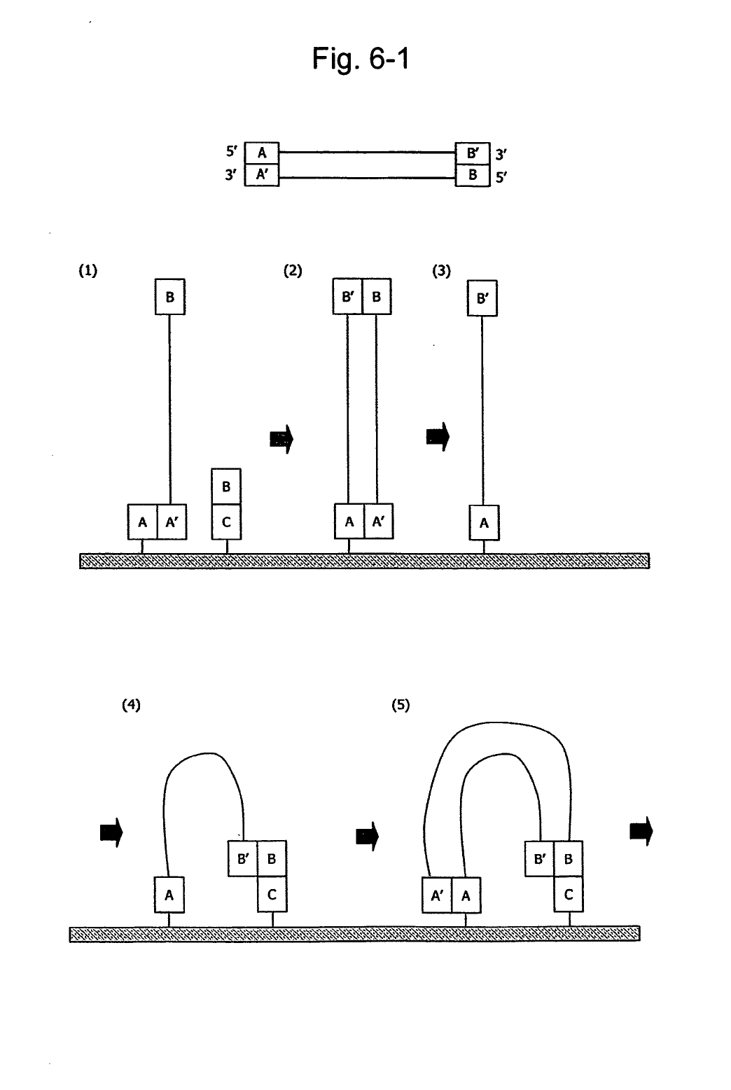

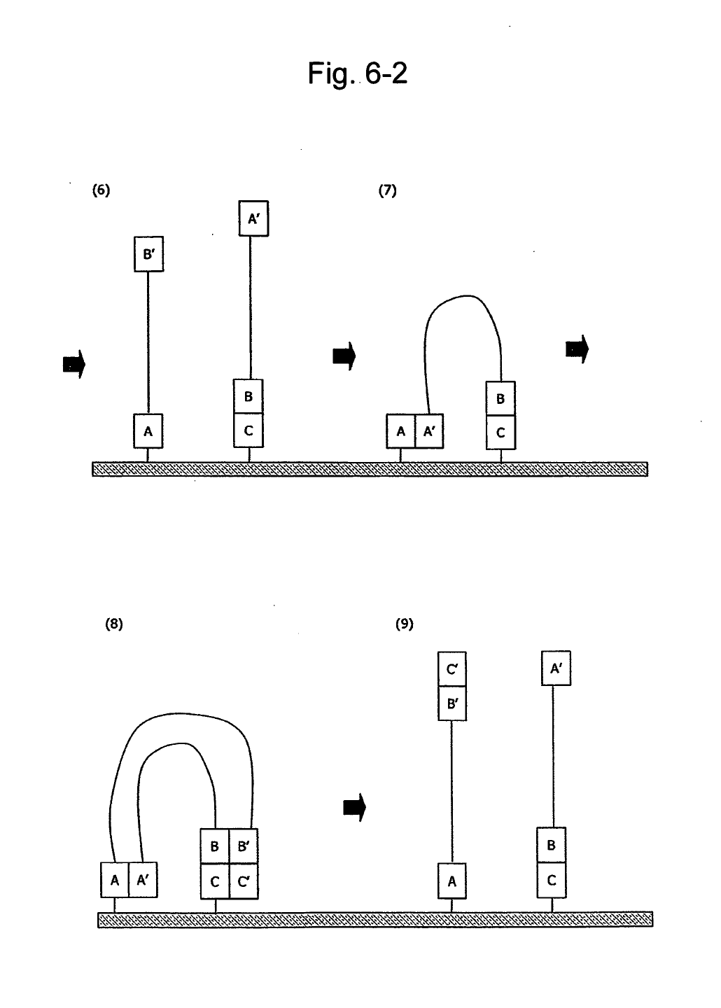

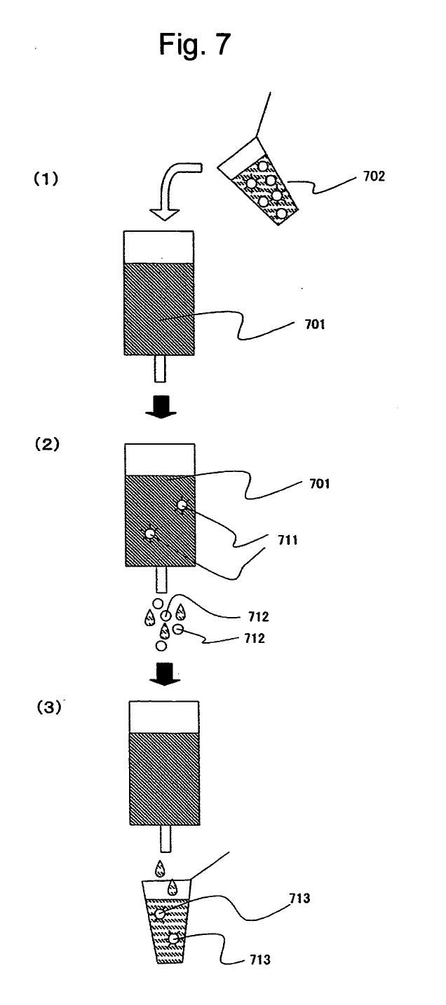

In the present Example, an anchor sequence for separation was added as separation means to a probe sequence introduced at the terminus of the amplified product, and the separation was performed by use of a column bound with a probe complementary to this anchor sequence (FIGS. 6 and 7). A probe 1 having a sequence A and a probe 2 having sequences B and C are immobilized on bead surface (FIG. 6-1(1)). As shown in FIG. 6, an adaptor 1 having a sequence A and a sequence A′ complementary to the sequence A and an adaptor 2 having a sequence B and a sequence B′ complementary to the sequence B are ligated to both termini of analyte DNA fragments. As shown in FIG. 6-1(1), one strand of the DNA fragment is complementarily annealed to the probe 1 on the bead surface, going into extension reaction (FIG. 6-1(2)). The DNA strand used as a template is denatured and washed (FIG. 6-1(3)). In the subsequent amplification step, a complementary strand extension product is complementarily annealed to the probe 2 on the bead surface (FIG. 6-1(4), going into extension reaction (FIG. 6-1(5)). After heat denaturing (FIG. 6-2(6)), each extension product is complementarily annealed to its nearest probe, going into extension reaction (FIGS. 6-2(7) and 6-2(8)). Finally, probe extension products produced on the bead surface are mainly strands having the sequence of FIG. 6-2(9). On the other hand, a probe having a sequence identical to a sequence C is immobilized in advance in a separation/purification column 701 shown in FIG. 7. To this column 701, beads 702 after amplification reaction are added, whereby a C′ sequence portion located at the terminus of an amplified product, if any, on the bead is complementarily annealed to the probe in the column 701 to capture the bead 711 (FIG. 7(2)). By contrast, beads with no amplified products do not have such a sequence capable of being complementarily annealed to the sequence C and therefore pass through the column 701 without being captured (beads represented by reference numeral 712 in FIG. 7(2)). After the addition of the bead solution, the column is washed with a buffer (10 mM Tris (pH 7.5)) with a low salt concentration to denature the complementary annealing. Eluted beads 713 are collected (FIG. 7(3)).

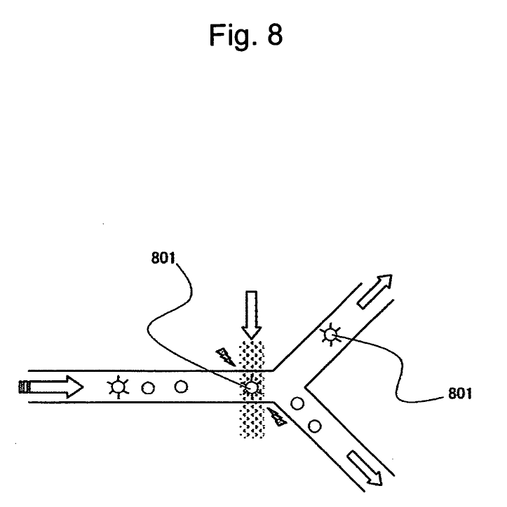

Another means for collecting only the bead bound with amplified products will be described (FIG. 8). A fluorescent dye (intercalator) capable of being specifically intercalated into the double-stranded portion of DNA was added during amplification reaction, or beads were suspended after amplification reaction in a solution containing the intercalator, whereby the double-stranded portions of amplified products on the bead were allowed to incorporate therein the fluorescent dye. The intercalator may be Pico Green (Invitrogen) or SYBR Green (Invitrogen). From this solution, only a bead 801 that emits a fluorescence was detected and collected by use of a flow cytometer, whereby only the bead 801 with amplified products was collected (FIG. 8).

### Example 4

In a pretreatment step of single-molecule measurement, it is very difficult to individually isolate each of molecules from a mixture of plural kinds of DNA samples. This approach may permit one bead-one DNA molecule binding. However, beads are hardly bound with DNA under reaction conditions for achieving this binding. Therefore, the bead bound with DNA must be separated. On the other hand, the single-molecule measurement which performs measurement without amplifying DNA samples is advantageous in that this approach counters the concern that amplification may not accurately reflect the sequences or quantitative ratio of the original nucleic acids. Thus, an approach using, as analytes, DNA strands obtained as complementary strand extension products in the initial step of the present invention and using amplified products as flags for bead separation will be described with reference to Example below.

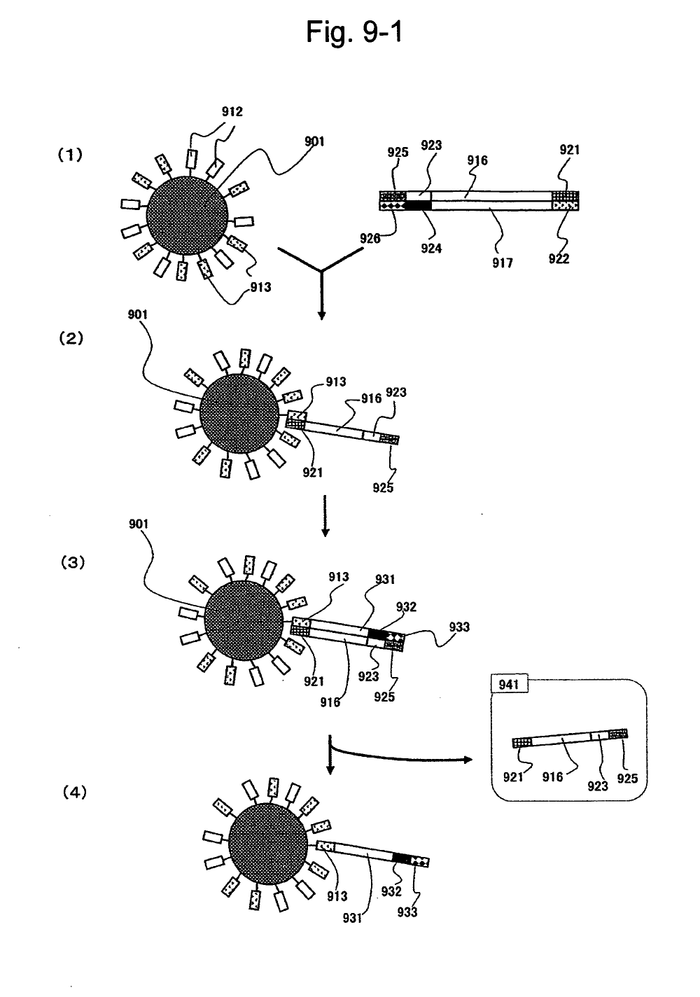

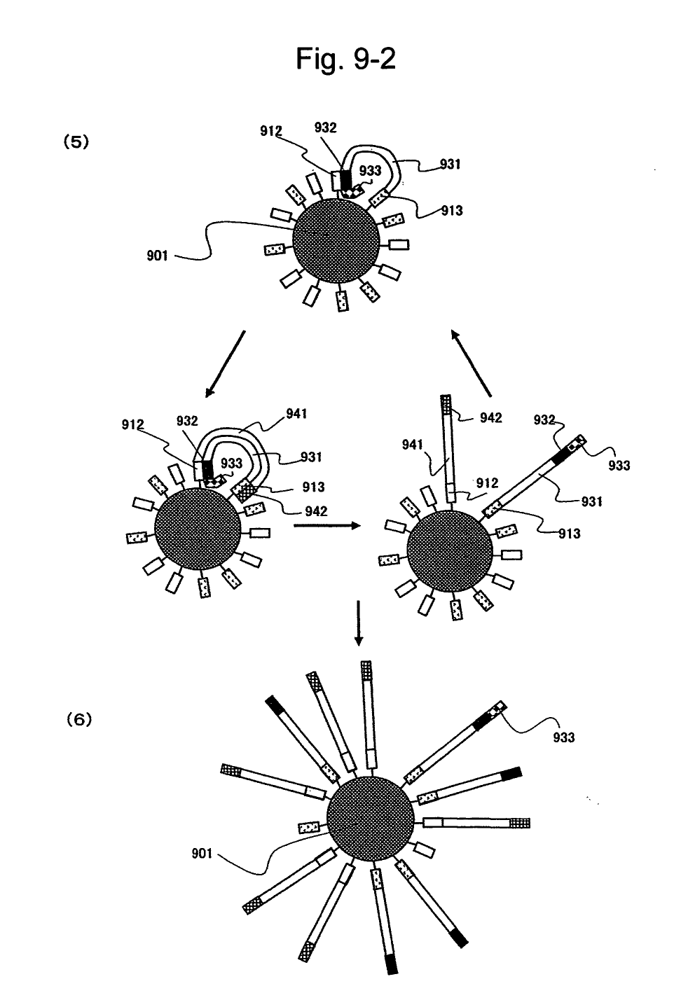

The steps of the present Example are schematically shown in FIG. 9. Magnetic beads (2.8 μm in diameter; Dynal BIOTECH) activated with carboxylic acid groups were used as solid phase carriers. 50 μL (1×10⁸ beads) of a solution containing the carboxylic acid group-activated magnetic beads well suspended in advance was measured into a 2.0 mL microtube. Magnets were placed on the side wall of the tube, and the supernatant was removed with the magnetic beads captured. To wash the beads, 100 μL of a MES Buffer (25 mM MES (pH 6.0), 0.1% (w/v) Tween 20) was added thereto, and the mixture was stirred and shaken at room temperature for 10 minutes. Then, the supernatant was removed. This step was repeated again, and the supernatant was removed. Subsequently, 30 μL each of solutions of probes A and B diluted to 2.5 μpmol/μL with a MES buffer was added thereto, and the mixture was stirred and shaken at room temperature for 30 minutes. Then, 30 μL of an EDC solution (adjusted to 0.1 mg/μL with a MES Buffer) and 10 μL of a MES buffer were added thereto, and the mixture was stirred and shaken overnight under conditions of 4° C. to immobilize the probes onto the magnetic beads. After the completion of reaction, the supernatant was removed. The carboxylic acid groups unbound with the probe were blocked. Specifically, 200 μL of a Blocking Buffer (50 mM Tris (pH 7.5), 0.1% (w/v) Tween 20) was added thereto, and the mixture was stirred and shaken at room temperature for 15 minutes. Then, the supernatant was removed. This step was repeated 4 times. Then, 200 μL of a 10 mM Tris (pH 7.5)-0.1% (w/v) Tween 20 solution was added thereto to obtain probe-immobilized beads at a final concentration of 5×10⁵ beads/μL. In FIG. 9-1, reference numeral 901 denotes a magnetic bead; reference numeral 912 denotes a probe A immobilized on the magnetic bead; and reference numeral 913 denotes a probe B immobilized on the magnetic bead. To easily understand the constitution of reaction, only one bead is shown in FIG. 9. However, 1×10⁸ beads actually coexist, as described above.

Subsequently, analyte DNA samples were prepared. The DNA samples are a mixture of double-stranded DNA fragments of approximately some hundreds bases to 1 kb in base length. An adaptor A having sequences 921 and 922 and an adaptor C having sequences 923, 924, 925, and 926 were ligated in advance to both termini of the double-stranded DNA fragments 916 and 917. The base sequence 922 constituting the adaptor A is wholly or partially complementary to the base sequence 921 and is wholly or partially identical to the probe B 913 immobilized on the magnetic bead 901. The base sequence 923 constituting the adaptor C is wholly or partially complementary to the base sequence 924 and is wholly or partially identical to the probe A 912 immobilized on the magnetic bead 901. The base sequence 925 constituting the adaptor C and the base sequence 926 wholly or partially complementary to the base sequence 925 are neither complementary nor identical to any of the probes 912 and 913 immobilized on the magnetic bead and the adaptors 921, 922, 923, and 924 ligated with the DNA sample.

Subsequently, the probe A (912)- and the probe B (913)-immobilized magnetic beads 901 were mixed with the double-stranded DNA fragments ligated with the adaptor A (921 and 922) and the adaptor C (923, 924, 925, and 926) to complementarily anneal the probes on the magnetic beads and the DNA fragments. 5×10² molecules of double-stranded DNA fragments were mixed for 10⁶ beads to perform reaction (to easily understand the constitution of reaction, only one kind of DNA fragment is shown in FIG. 9. However, 5×10² molecules of DNA fragments actually coexist). The mixed solution of the beads and the DNA fragments was mixed with 4 μL of a 10×PCR buffer (600 mM Tris-SO₄ (pH 8.9), 180 mM Ammonium Sulfate), 1.6 μL of 50 mM MgSO₄, 0.8 μL of a 10 mM dNTP Mix (mixed solution of dATP, dCTP, dGTP, and dTTP), and 0.4 μL of Platinum Taq DNA Polymerase High Fidelity (5 units/μL), and the total amount of the solution was adjusted to 40 μL with distilled water (DW) to prepare a reaction solution. Subsequently, the DNA fragments were completely denatured into single strands at 94° C. for 60 seconds. Subsequently, complementary strand formation and extension reaction were performed by incubation at 50° C. for 120 seconds→at 72° C. for 120 seconds. In this step, the adaptor 921 ligated to the terminus of the DNA molecule (composed of 925, 923, 916, and 921) was complementarily annealed to the probe B 913 on the bead such that the probe B 913 was extended with the DNA molecules 916, 923, and 925 as templates (FIGS. 9-1(2) and 9-1(3)).

Subsequently, to remove the single-stranded DNA sample used as a template in complementary annealing/complementary strand extension and the DNA sample adsorbed on the bead surface, the microtube was washed with solutions of (i) 0.5 N NaOH (room temperature, 1 min.×two times), (ii) 1×TE (94° C., 1 min.×one time), and (iii) 10 mM Tris (pH 7.5) (94° C., 1 min.×one time). The supernatant was removed. The washing solutions may be supplemented, if necessary, with Tween 20 at a final concentration of approximately 0.01 to 0.1% (w/v) to prevent bead aggregation or adsorption onto the internal wall of a pipette chip. This washing/removal step is essential for achieving one bead-one DNA molecule. Without this step, the residual DNA sample forms second complementary strand extension products with the probe on the bead already having complementary strand extension products in the subsequent amplification step, resulting in amplified products of one bead-plural kinds of DNAs. The 3′-terminus of the complementary strand extension product remaining on the bead after washing were ligated with a portion 933 having a sequence identical to the base sequence 926 constituting the adaptor C.

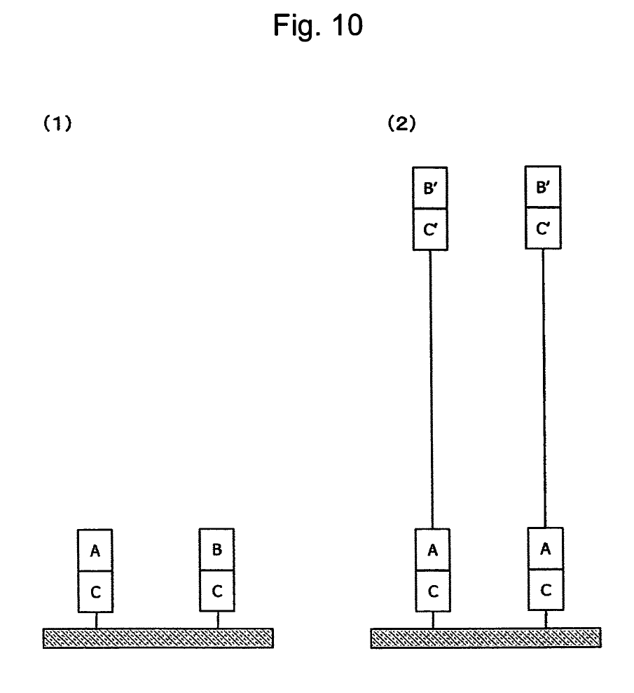

Subsequently, the complementary strand extension products on the bead after washing were amplified. The washed beads (10⁶) were mixed with 4 μL of a 10×PCR buffer (600 mM Tris-SO₄ (pH 8.9), 180 mM Ammonium Sulfate), 1.6 μL of 50 mM MgSO₄, 0.8 μL of 10 mM dNTP, and 0.4 μL of Platinum Taq DNA Polymerase High Fidelity (5 units/μL), and the total amount of the solution was adjusted to 40 μL with DW to prepare a reaction solution. 50 cycles each involving 94° C. for 30 seconds (heat denaturing of DNA into single strands)→55° C. for 120 seconds (complementary annealing to the probe immobilized on the bead)→72° C. for 45 seconds (complementary strand extension reaction of the probe) were performed (FIG. 9-2(5)). Finally, the temperature was set to 72° C. for 10 minutes and then lowered to room temperature. In this amplification step, the complementary strand extension products (931, 932, and 933) of the probe immobilized on the bead in the initial step function as templates. The sequence portion 932 of this complementary strand extension product (931, 932, and 933) formed a complementary strand with the probe A 912 immobilized on the bead to extend the probe A 912, whereby a new DNA strand (complementary strand extension products 941 and 942 of the probe) was formed on the bead. In this procedure, the DNA as a template on the solid phase forms a complementary strand in a bent form such that the complementary strand forms a U shape with its neighboring probe on the same bead. The extension product also takes a bent form. In the step of heat denaturing into single strands, this bent form is heat denatured to form a single-stranded DNA immobilized on the bead, which is then used as a template in a next cycle. Finally, a large number of amplified products are obtained on the bead (FIG. 9-2(6)). To achieve one bead-one kind of DNA sample, a reaction solution containing 10³ or less DNA molecules must be prepared, as described in FIG. 2, whereby the number of a bead comprising two or more molecules immobilized thereon can be one or less in a reaction system using 10⁶ beads. According to this calculation, 998,400 beads corresponding to 98.4% of the whole are bound with no DNA fragments. Thus, only the bead with obtained amplified products was separated and collected by use of any of the methods described in Example 3. When this separation is performed by use of the column shown in FIG. 7, the sequence of a probe immobilized in the column comprises a sequence C for capturing added to the terminus of the immobilized probe 912 or 913 shown in FIG. 9 (FIG. 10(1)). In amplification using this probe, the terminal region of a main amplified product different from the terminus immobilized on the bead surface is ligated with a sequence C′ complementary to the sequence C (FIG. 10(2)). If a probe having the sequence C is immobilized in the column, only the bead with amplified products, that is, only the bead having the sequence C′ is captured by the column, whereas the beads with no amplified products pass through the column. In this way, only the bead with amplified products can be separated and collected.

Meanwhile, the amplified products produced on the bead differ in their sequences between the complementary strand extension products obtained in the initial step and the amplified products obtained in the subsequent step. Only the termini of the initial extension products have a sequence portion 933 having a sequence identical to the base sequence 926 constituting the adaptor C, and being neither complementary nor identical to the probe sequence on the bead is added thereto. DNA base sequence determination is performed according to a well-known method using primers having a sequence complementary to the sequence portion 933 obtained by the complementary strand extension of this original DNA molecule, whereby DNA analysis of one bead-one molecule, not amplified products, can be achieved. Specifically, the single-molecule measurement of the original DNA without bias such as amplification was achieved by isolating DNA samples provided as a mixture by use of the amplified products on the bead and analyzing initial complementary strand extension products.

### Example 5

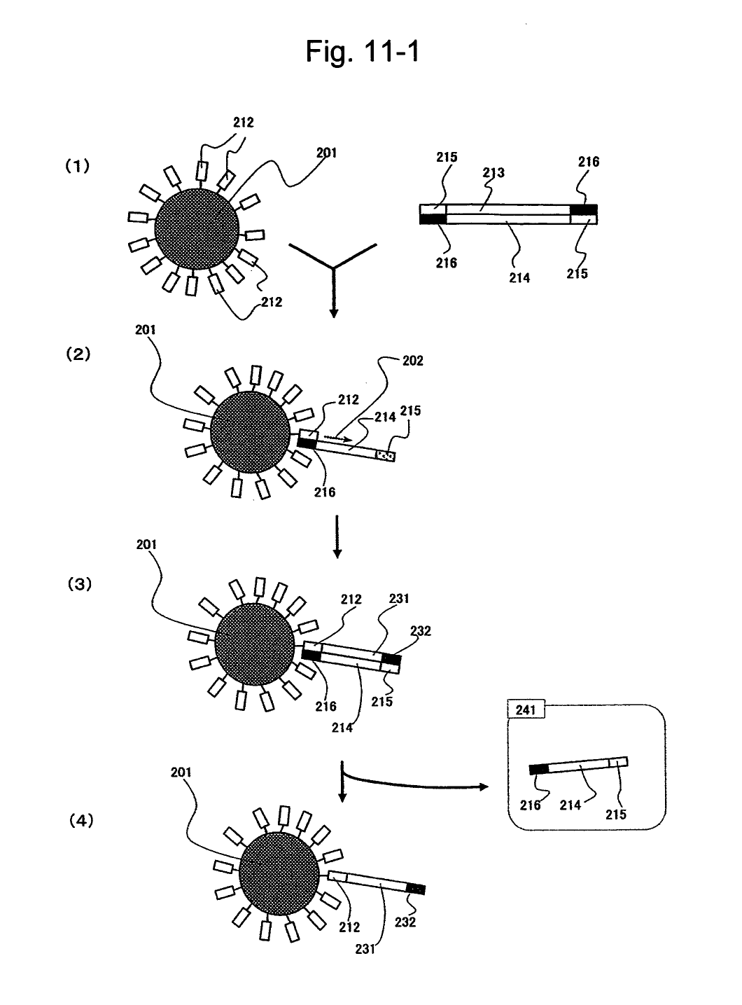

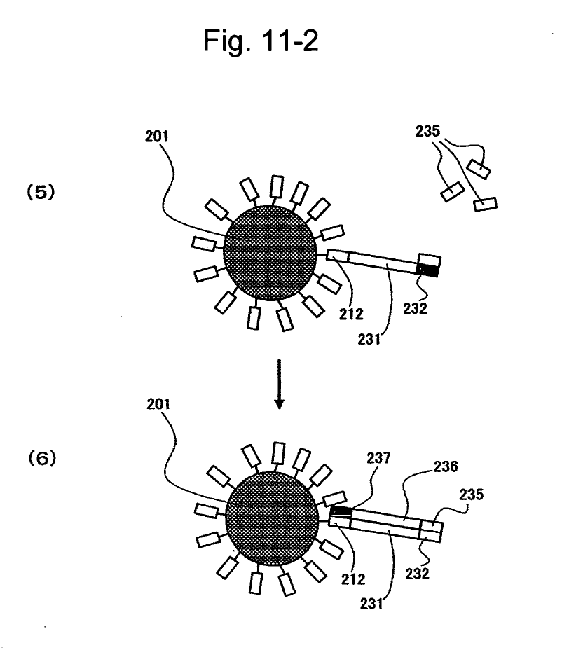

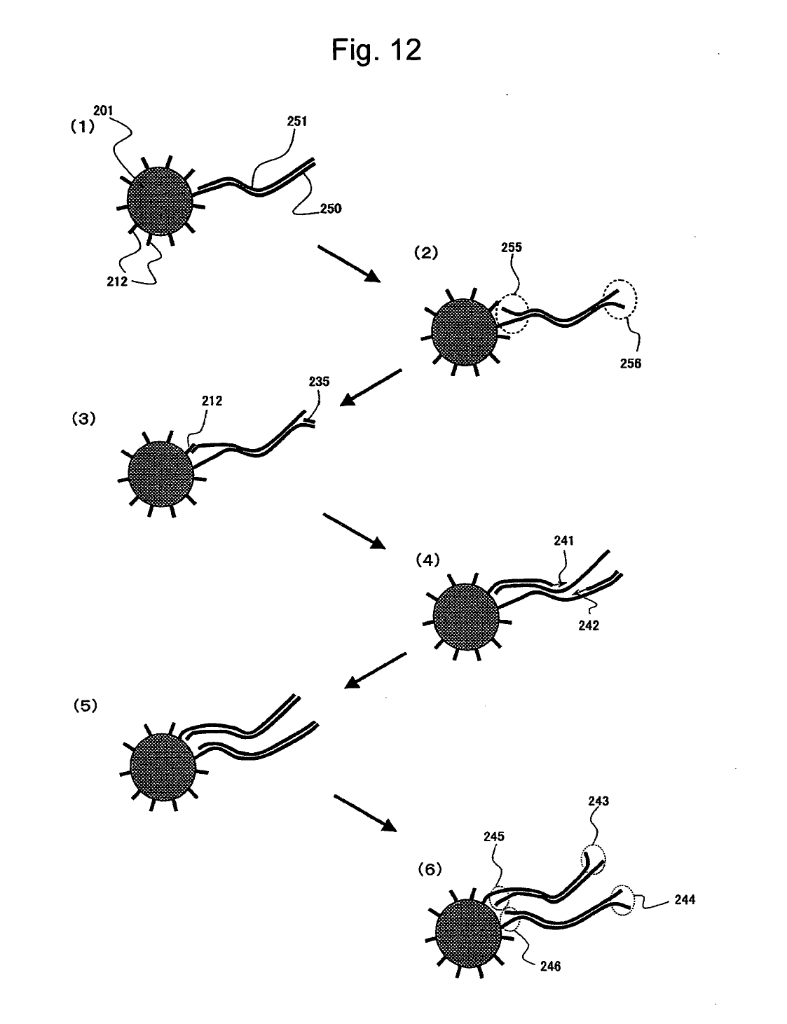

To achieve individual and parallel amplification based on one bead-one DNA, amplification probes are not necessarily required to be immobilized on beads, when amplified products can move only in the very near neighborhood of the bead. This Example shows a nucleic acid analysis method which is characterized in that: (1) an adaptor having a sequence complementary to the immobilized probe is ligated to the terminal region of a nucleic acid sample as a template, and an adaptor region having a sequence identical to a suspended probe is ligated to the other terminus thereof; (2) the nucleic acid sample as a template is capable of being complementarily annealed to the immobilized probe; (3) an extension product of the immobilized probe complementarily annealed therewith is capable of being complementarily annealed to the suspended probe; and (4) only the terminal regions of a double-stranded nucleic acid sample comprising the extension products of the immobilized and suspended probes are partially denatured. The present Example will be described with reference to FIGS. 11 and 12.

Sepharose beads (approximately 34 μm in diameter; GE Healthcare Bioscience) activated with streptavidin groups were used as solid phase carriers. A probe A was immobilized thereon. The 5′ terminal region of the probe A used here is modified with biotin capable of binding with streptavidin. Eighteen carbon molecules are inserted as a spacer between the biotin modification and the base sequence. 100 μL (1×10⁶ beads) of the streptavidin group-activated Sepharose beads well suspended in advance was added to a spin column (Ultra free-MC, durapore PVDF 0.5 μm; Millipore) and centrifuged at 12,000×g for 1 minute. The supernatant was removed. The beads on the column were suspended with 150 μL of a 2×Binding Buffer (10 mM Tris-HCl, 1 mM EDTA, 2 M NaCl, 0.01% (w/v) Tween 20), and this suspension was transferred to a 2.0 mL microtube. Subsequently, 50 μL of a solution of 10 μpmol/μL probe A was added thereto, and the total amount of the solution was adjusted to 300 μL with distilled water (DW). The solution was mixed under room temperature conditions for 1 hour by use of a rotator to bind the streptavidin on the bead to the biotin group at the terminus of the probe. The efficiency of binding with streptavidin can be enhanced by modification using dual biotin groups comprising two consecutive biotin groups, instead of the biotin groups. In FIG. 11(1), reference numeral 201 denotes a Sepharose bead, and reference numeral 212 denotes a probe A immobilized through streptavidin-biotin binding. For the sake of simplification, only one bead is shown in the drawing. Subsequently, analyte DNA samples were prepared. The DNA samples are a mixture of double-stranded DNA fragments of approximately some hundreds bases to 1 kb in base length. In FIG. 11-1(1), reference numerals 213 and 214 denote a DNA sample. For the sake of simplification, only one fragment is shown as a DNA strand in the drawing. An adaptor A having sequences 215 and 216 was ligated in advance to both termini of the double-stranded DNA fragments 213 and 214. The base sequence 215 constituting the adaptor A is wholly or partially complementary to the base sequence 216 and is wholly or partially identical to the probe A 212 immobilized on the Sepharose bead 201.

Subsequently, the probe A (212)-immobilized Sepharose bead 201 was mixed with the double-stranded DNA fragments ligated with the adaptor A (215 and 216) to complementarily anneal the probes on the Sepharose bead and the DNA fragments. Three tubes containing 1×10⁵, 1×10⁴, or 1×10³ molecules (respectively corresponding to 10, 1, or 0.1 molecules per bead) of double-stranded DNA fragments mixed for 10⁴ beads were prepared to perform reaction (FIG. 11-1(2)). The mixed solution of the beads and the DNA fragments was mixed with 2 μL of a 10×PCR buffer (600 mM Tris-SO₄ (pH 8.9), 180 mM Ammonium Sulfate), 0.8 μL of 50 mM MgSO₄, 0.4 μL of a 10 mM dNTP Mix (mixed solution of dATP, dCTP, dGTP, and dTTP), and 0.4 μL of Platinum Taq DNA Polymerase High Fidelity (5 units/μL), and the total amount of the solution was adjusted to 20 μL with distilled water (DW) to prepare a reaction solution. Subsequently, the DNA fragments were completely denatured into single strands at 94° C. for 60 seconds. Subsequently, complementary strand formation and extension reaction were performed by incubation at 50° C. for 120 seconds→at 72° C. for 120 seconds. In this step, the adaptor 216 ligated to the terminus of the DNA molecule (composed of 216, 214, and 215) was complementarily annealed to the probe A 212 on the bead such that the probe A 212 was extended in a direction 202 with the DNA molecules 214 and 215 as templates (FIGS. 11-1(2) and 11-1(3)). In FIG. 11-1, reference numerals 231 and 232 denote a complementary strand extension product of the probe. The terminus of the extension product was ligated with the portion 232 having a sequence complementary to the probe A 212 immobilized on the bead. Subsequently, to remove the single-stranded DNA sample (composed of 216, 214, and 215) used as a template in complementary annealing/complementary strand extension, the DNA sample adsorbed on the bead surface, and the redundant DNA sample that had not participated in binding, the microtube was washed with solutions of (i) 0.5 N NaOH (room temperature, 1 min.×two times), (ii) 1×TE (94° C., 1 min.×one time), and (iii) 10 mM Tris (pH 7.5) (94° C., 1 min.×one time). The supernatant was removed (In FIG. 11-1, DNA samples boxed within a broken line 241). The removal of the supernatant was performed by the following step: the Sepharose beads were precipitated to the bottom of the tube by centrifugation, and the supernatant was gently removed with a pipette. After the addition of a washing solution, the solution was sufficiently stirred by use of vortex or the like, and the supernatant was removed again (FIG. 11-1(4)).

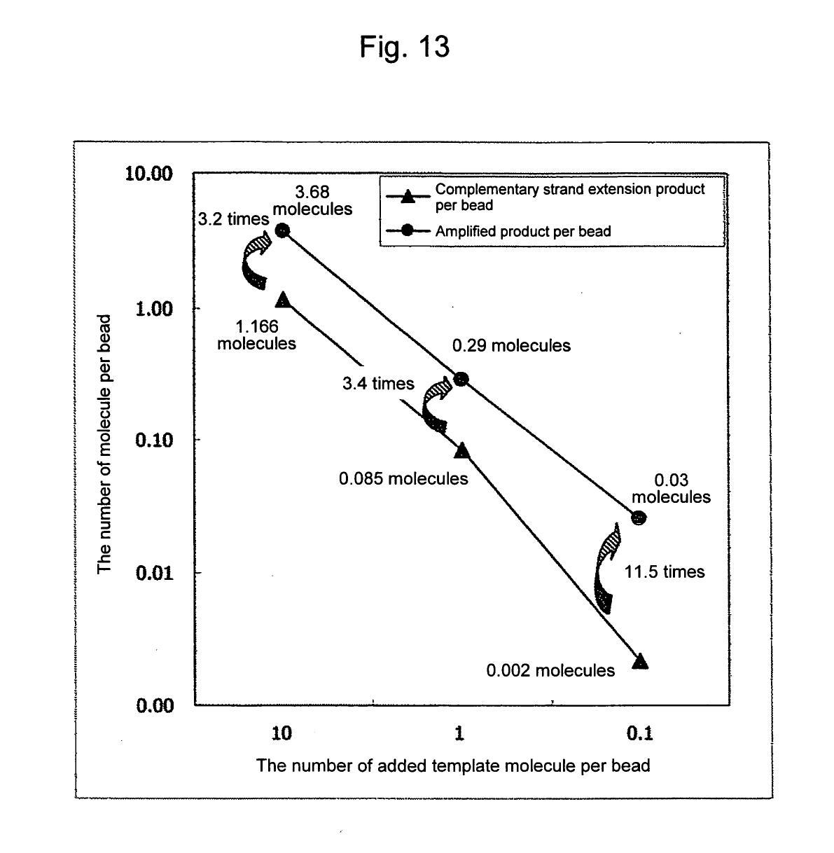

Subsequently, the complementary strand extension products on the bead after washing were amplified. The washed beads (10⁴) were mixed with 2 μL of the primer A diluted to 10 μpmol/μL with DW, 2 μL of a 10×reaction solution (200 mM Tris-HCl (pH 8.8), 100 mM KCl, 100 mM (NH₄)₂SO₄, 20 mM MgSO₄, 1.0% Triton X-100), 1 μL of 10 mM dNTP, and 1 μL of Bst DNA Polymerase (8 units/μL) (New England Biolabs), and the total amount of the solution was adjusted to 20 μL with DW to prepare a reaction solution. The beads were suspended in this solution. The Bst DNA Polymerase used here is capable of strand displacement. Even when an extended strand after the complementary annealing between the template DNA and the amplification primer is used as a double-stranded template in the direction of extension, this enzyme is capable of synthesizing a complementary strand while denaturing the double strand. In the present Example, the Bst DNA Polymerase was used. However, DNA polymerase used in this procedure is not limited to Bst DNA Polymerase, and any DNA polymerase capable of strand displacement, such as Deep Vent_{R} DNA Polymerase (New England Biolabs) or 9Nₘ™ DNA Polymerase (New England Biolabs), can be expected to have the same effects. Then, the reaction solution was kept at 65° C. to perform reaction for 90 minutes. Then, the enzyme was inactivated at 94° C. for 2 minutes. The primers A (235) diffused in the solution were complementarily annealed to the terminal regions 232 of the complementary strand extension products on the bead under constant temperature conditions of 65° C. (FIG. 11-2(5)) such that extension reaction occurred to obtain products (represented by 235, 236, and 237 in FIG. 11-2(6)). For the sake of simplifying drawings, the initial complementary strand extension products (212, 231, and 232) in FIG. 11-2 are represented by reference numeral 250 in FIG. 12, and extension products (235, 236, and 237) having a sequence complementary to the initial complementary strand extension product are represented by reference numeral 251 in FIG. 12. Under the conditions of 65° C., only the terminal portions 255 and 266 of the produced double-stranded DNA are partially denatured. This partially denatured single-stranded portion 256 is complementarily annealed to the primer A (235) diffused in the solution. The other terminal region 255 is complementarily annealed to the probe A (212) on the bead located in the neighborhood of the terminal region (FIG. 12(3)). Then, extension reaction proceeds in directions 241 and 242, while the double-stranded complementarily annealed portion as a template was denatured (FIG. 12(4)). As a result, products shown in FIG. 12(5) are obtained. Likewise, under the conditions of 65° C., only the terminal portions of the products are partially denatured. Therefore, denatured portions 243 and 244 are complementarily annealed to the primer A diffused in the reaction solution. Denatured portions 245 and 246 of the other terminal region are complementarily annealed to their nearest immobilized probes A on the bead, going into new extension reaction. Finally, plural complementary strand extension products could be obtained on the bead under the constant temperature conditions. The results are shown in FIG. 13. The term “Complementary strand extension product per bead” in the drawing corresponds to FIG. 11(4) and refers to the number of a molecule used as a template in amplification. On the other hand, the term “Amplified product per bead” refers to the number of a molecule after the amplification of the extension product. Under the reaction conditions described in the present Example, the best results were obtained in a reaction system obtained by adding DNA molecules in a 1/10 amount of 10⁴ beads. In this case, the molecules could be amplified by 11.5 times.

Under usual PCR reaction conditions, denaturing is performed at approximately 94° C. Therefore, the double-stranded structures of complementary strand extension products are completely denatured. The strand having a non-immobilized terminus is diffused into the solution. However, complementary strand extension products are not completely denatured by using an enzyme capable of strand displacement, as in the present Example. Therefore, these complementary strand extension products are neither separated from the bead nor diffused into the solution. Such products contribute to reaction only on their initially bound bead until the final stage.

Even when PCR reaction is performed by use of a usual PCR enzyme, for example, a solvent (e.g., Mebiol Gel™ (Mebiol Inc.) which is characterized by being in a gel state at a transition temperature or higher and in a flowable sol state at a transition temperature or lower) or methylcellulose gel which has a viscosity increased under high temperature conditions is added to a reaction solution, whereby amplified products of a probe having a non-immobilized terminus can move only in the very near neighborhood of the bead surface. In this case, complementary strand extension products are neither separated from the bead nor diffused into the solution. Thus, the same effects as in the present Example can be obtained.

In this way, individual and parallel amplification based on one bead-one DNA could be achieved.

What is claimed is:

1. A nucleic acid analysis method for simultaneously analyzing plural nucleic acid samples, comprising:
a first step of introducing plural template nucleic acids to plural solid phase carriers such that one solid phase carrier comprising one or more kinds of amplification probes immobilized on the surface is capable of being bound via the probe to a terminal region comprising the 3′ terminus of one template nucleic acid molecule;
a second step of extending the probe with the template nucleic acid as a template to form a first extended probe;
a third step of denaturing the template nucleic acid from the first extended probe;
a fourth step of removing the template nucleic acid;
a fifth step of repeating the steps of (1) annealing a terminal region comprising the 3′ terminus of the extended probe to an unextended probe, (2) extending the unextended probe with the first extended probe as a template to form a second extended probe, and (3) denaturing the first extended probe from the second extended probe, whereby the first and second extended probes are amplified to form a large number of the first and second extended probes on the carrier; and
a sixth step of separating the carrier bound with the first extended probes from the carrier unbound with the first extended probes.

2. The nucleic acid analysis method according to claim 1, further comprising, before the first step, the step of ligating an adaptor having a first sequence to the 3′ termini of the template nucleic acids and ligating an adaptor having a second sequence different from the first sequence to the 5′ termini of the template nucleic acids, wherein
each of the plural probes immobilized on the one carrier has a complementary sequence to either the first or second sequence.

3. The nucleic acid analysis method according to claim 1, further comprising, before the first step, the step of ligating an adaptor having a first sequence to the 3′ termini of the template nucleic acids and ligating an adaptor having a complementary sequence to the first sequence to the 5′ termini of the template nucleic acids, wherein
each of the plural probes immobilized on the one carrier has a complementary sequence to the first sequence.

4. The nucleic acid analysis method according to claim 1, wherein the first to fourth steps are performed in the same container, and the fifth step is performed in different containers individually accommodating each of the plural carriers.

5. The nucleic acid analysis method according to claim 1, wherein the first to fifth steps are performed in different containers individually accommodating each of the plural carriers.

6. The nucleic acid analysis method according to claim 4, wherein a solution for performing the reaction is common to the different containers individually accommodating each of the plural carriers.

7. The nucleic acid analysis method according to claim 1, wherein the fifth step comprises repeating the steps of (1) extending a complementary strand with the first extended probe as a template to form a second extended probe in a bent form such that the complementary strand forms a U shape with its neighboring probe on the same solid phase carrier, and (2) heat denaturing the bent form to form a single-stranded nucleic acid immobilized on the carrier, which is then used as a template in a next cycle.

8. The nucleic acid analysis method according to claim 1, wherein in the first to fifth steps, the reaction solution is constantly stirred.

9. The nucleic acid analysis method according to claims 1, wherein in the first to fifth steps, the plural carriers are located at a distance longer than the length of the template nucleic acid from each other.

10. A nucleic acid analysis method for simultaneously analyzing plural nucleic acid samples, comprising:
a first step of introducing plural template nucleic acids to plural solid phase carriers such that one solid phase carrier comprising one kind of probes immobilized on the surface is capable of being bound via the probe to a terminal region comprising the 3′ terminus of one template nucleic acid molecule;
a second step of extending the immobilized probe with the template nucleic acid as a template to form a first extended probe;
a third step of denaturing the template nucleic acid from the first extended probe;
a fourth step of removing the template nucleic acid;
a fifth step of repeating the steps of (1) annealing a terminal region comprising the 3′ terminus of the first extended probe to another kind of suspended probe added to the reaction solution, (2) extending the suspended probe with the first extended probe as a template to form a second extended probe, and (3) denaturing the first extended probe from the second extended probe, whereby the first and second extended probes are amplified to form a large number of the first and second extended probes on the carrier; and
a sixth step of separating the carrier bound with the first extended probes from the carrier unbound with the first extended probes.

11. The nucleic acid analysis method according to claim 10, wherein the step (3) in the fifth step comprises partially denaturing only the terminal regions of a double-stranded nucleic acid composed of the first and second extended probes to form single-stranded terminal regions, complementarily annealing the single-stranded terminal regions to the immobilized probe or the suspended probe, and performing extension reaction while denaturing the double-stranded portion of the template nucleic acid by use of DNA polymerase capable of strand displacement.

12. The nucleic acid analysis method according to claim 10, wherein a substance which has an increased viscosity or is gelled during denaturing and has a decreased viscosity or is in a solution state during complementary annealing is allowed to coexist in the reaction solution, and
after the capturing of the template nucleic acid by the carrier, the nucleic acid amplification reaction is performed in a state where the suspended probes are dispersed in gel.

13. The nucleic acid analysis method according to claim 1, wherein the first to fifth steps are performed, during which an anchor sequence for separation which is neither complementary nor identical to the first and second sequences of the adaptors is added to a probe sequence annealed to the 5′ terminus of the template nucleic acid, and wherein, in the sixth step, only the solid phase carrier with obtained amplified products is separated by use of a column bound with a probe complementary to the anchor sequence.

14. The nucleic acid analysis method according to claim 1, wherein the first to fourth steps are performed, during which a third sequence which is neither complementary nor identical to the first and second sequences of the adaptors is added to a probe sequence annealed to the 5′ terminus of the template nucleic acid, and wherein, the method further comprises the step of sequencing the template nucleic acid which is not an amplified product by use of a primer having a sequence complementary to the third sequence.

15. The nucleic acid analysis method according to claim 1, wherein only the solid phase carrier with obtained amplified products is separated by adding a double strand-specific intercalator to the amplification reaction solution or to a solid phase carrier suspension after the completion of amplification reaction and detecting/collecting only the solid phase carrier that emits a fluorescence derived from the intercalator from the solution.

16. The nucleic acid analysis method according to claim 1, wherein a reaction solution comprising 10³ or less template nucleic acid molecules for a reaction system using 10⁶ solid phase carriers is prepared to prevent amplified products attributed to two or more template nucleic acids from being replicated on one solid phase carrier.

17. The nucleic acid analysis method according to claim 1, wherein the amplification reaction is performed in a solution comprising a homogeneous solvent.

18. The nucleic acid analysis method according to claim 1, wherein the solid phase carriers are beads.

19. The nucleic acid analysis method according to claim 5, wherein a solution for performing the reaction is common to the different containers individually accommodating each of the plural carriers.

20. The nucleic acid analysis method according to claim 10, wherein the first to fifth steps are performed, during which an anchor sequence for separation which is neither complementary nor identical to the first and second sequences of the adaptors is added to a probe sequence annealed to the 5′ terminus of the template nucleic acid, and wherein, in the sixth step, only the solid phase carrier with obtained amplified products is separated by use of a column bound with a probe complementary to the anchor sequence.

21. The nucleic acid analysis method according to claim 10, wherein the first to fourth steps are performed, during which a third sequence which is neither complementary nor identical to the first and second sequences of the adaptors is added to a probe sequence annealed to the 5′ terminus of the template nucleic acid, and wherein, the method further comprises the step of sequencing the template nucleic acid which is not an amplified product by use of a primer having a sequence complementary to the third sequence.

22. The nucleic acid analysis method according to claim 10, wherein only the solid phase carrier with obtained amplified products is separated by adding a double strand-specific intercalator to the amplification reaction solution or to a solid phase carrier suspension after the completion of amplification reaction and detecting/collecting only the solid phase carrier that emits a fluorescence derived from the intercalator from the solution.

23. The nucleic acid analysis method according to claim 10, wherein a reaction solution comprising 10³ or less template nucleic acid molecules for a reaction system using 10⁶ solid phase carriers is prepared to prevent amplified products attributed to two or more template nucleic acids from being replicated on one solid phase carrier.

24. The nucleic acid analysis method according to claim 10, wherein the amplification reaction is performed in a solution comprising a homogeneous solvent.

25. The nucleic acid analysis method according to claim 10, wherein the solid phase carriers are beads.
